(19) United States
(12) Patent Application Publication
(10) Pub. No.: US 2026/0350805 A1
(43) Pub. Date: Nov. 5, 2026

(54) MACHINE LEARNING ENABLED ANALYSIS OF MEDICAL IMAGING DATA FOR ASSESSMENT OF DISEASE PROGRESSION AND TREATMENT RESPONSE

(71) Applicant: Genentech, Inc., South San Francisco, CA (US)

(72) Inventors: Xiaoyong WANG, South San Francisco, CA (US); Richard Alan Duray CARANO, San Ramon, CA (US); Jill Osborn FREDRICKSON, San Francisco, CA (US); Mohammad Skander JEMAA, San Francisco, CA (US)

(21) Appl. No.: 19/713,165

(22) Filed: Jun. 18, 2026

Related U.S. Application Data
(63) Continuation of application No. PCT/US2024/060066, filed on Dec. 13, 2024.
(60) Provisional application No. 63/612,796, filed on Dec. 20, 2023.

(51) Int. Cl. G06T 7/00; A61B 6/00; A61B 6/03; A61B 6/50; G06T 7/11; G06T 7/33; G06T 7/73; G06V 10/75

(52) U.S. Cl. CPC G06T 7/0016; A61B 6/032; A61B 6/50; A61B 6/5205; A61B 6/5235; G06T 7/11; G06T 7/337; G06T 7/74; G06V 10/759; G06T 2207/10081; G06T 2207/10104; G06T 2207/30096; G06T 2207/30204; G06V 2201/03

(57) ABSTRACT

A method may include determining a first tumor mask identifying a first set of lesions present in a first positron emission tomography and computed tomography (PET/CT) scan from a first timepoint. A second tumor mask identifying a second set of lesions present in a second PET/CT scan from a second timepoint may be determined. A first matching lesion in the second tumor mask that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask may be identified based at least on the first tumor mask and the second tumor mask. A change in metabolic level between the first timepoint and the second timepoint may be determined based on at least a metabolic level of each of the first target lesion in the first PET/CT scan and the first matching lesion in the second PET/CT scan. A response to a treatment for a disease may be determined, based at least on the change in metabolic level between the first timepoint and the second timepoint.

### CROSS REFERENCE TO RELATED APPLICATION

This application claims priority to U.S. Provisional Application No. 63/612,796, entitled “MACHINE LEARNING ENABLED ANALYSIS OF MEDICAL IMAGING DATA FOR ASSESSMENT OF DISEASE PROGRESSION AND TREATMENT” and filed on Dec. 20, 2023, the disclosure of which is incorporated by reference in its entirety.

### TECHNICAL FIELD

The subject matter described herein relates generally to machine learning and more specifically to machine learning enabled techniques for assessing disease progression and treatment response based on medical imaging data.

### INTRODUCTION

Medical imaging refers to techniques and processes for obtaining data characterizing a subject's internal anatomy and pathophysiology including, for example, images created by the detection of radiation either passing through the body (e.g. x-rays) or emitted by administered radiopharmaceuticals (e.g. gamma rays from intravenously administered radioactive tracers). By revealing internal anatomical structures obscured by other tissues such as skin, subcutaneous fat, and bones, medical imaging is integral to numerous medical diagnosis and/or treatments. Examples of medical imaging modalities include 2-dimensional imaging such as x-ray plain films, bone scintigraphy, and thermography. Examples of 3-dimensional imaging modalities include magnetic resonance imaging (MRI), computed tomography (CT), cardiac sestamibi scanning, and positron emission tomography (PET).

### SUMMARY

Systems, methods, and articles of manufacture, including computer program products, are provided for machine learning enabled analysis of medical imaging data, such as positron emission tomography and computed tomography (PET-CT) scans, for assessing disease progression and treatment response. In one aspect, there is provided a system that includes at least one data processor and at least one memory. The at least one memory may store instructions, which when executed by the at least one data processor, result in operations including: determining a first tumor mask identifying a first set of lesions present in a first positron emission tomography and computed tomography (PET/CT) scan from a first timepoint; determining a second tumor mask identifying a second set of lesions present in a second PET/CT scan from a second timepoint; identifying, based at least on the first tumor mask and the second tumor mask, a first matching lesion in the second tumor mask that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the first PET/CT scan and the first matching lesion in the second PET/CT scan, a change in metabolic level between the first timepoint and the second timepoint; and determining, based at least on the change in metabolic level between the first timepoint and the second timepoint, a response to a treatment for a disease.

In another aspect, there is provided a computer-implemented method that includes: determining a first tumor mask identifying a first set of lesions present in a first positron emission tomography and computed tomography (PET/CT) scan from a first timepoint; determining a second tumor mask identifying a second set of lesions present in a second PET/CT scan from a second timepoint; identifying, based at least on the first tumor mask and the second tumor mask, a first matching lesion in the second tumor mask that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the first PET/CT scan and the first matching lesion in the second PET/CT scan, a change in metabolic level between the first timepoint and the second timepoint; and determining, based at least on the change in metabolic level between the first timepoint and the second timepoint, a response to a treatment for a disease.

In another aspect, there is provided a computer program product including a non-transitory computer readable medium. The non-transitory computer readable medium may store instructions, which when executed by at least one data processor, result in operations including: determining a first tumor mask identifying a first set of lesions present in a first positron emission tomography and computed tomography (PET/CT) scan from a first timepoint; determining a second tumor mask identifying a second set of lesions present in a second PET/CT scan from a second timepoint; identifying, based at least on the first tumor mask and the second tumor mask, a first matching lesion in the second tumor mask that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the first PET/CT scan and the first matching lesion in the second PET/CT scan, a change in metabolic level between the first timepoint and the second timepoint; and determining, based at least on the change in metabolic level between the first timepoint and the second timepoint, a response to a treatment for a disease.

In another aspect, there is provided a system that includes at least one data processor and at least one memory. The at least one memory may store instructions, which when executed by the at least one data processor, result in operations comprising: determining a first tumor mask identifying a first set of lesions present in a baseline positron emission tomography and computed tomography (PET/CT) scan captured at screening; determining a second tumor mask identifying a second set of lesions present in a followup PET/CT scan captured at followup; identifying, in the second tumor mask, a first matching lesion that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the baseline PET/CT scan and the first matching lesion in the followup PET/CT scan, a change in metabolic level between screening and followup; and determining, based at least on the change in metabolic level between screening and followup, a response to a treatment for a disease.

In another aspect, there is provided a method that includes: determining a first tumor mask identifying a first set of lesions present in a baseline positron emission tomography and computed tomography (PET/CT) scan captured at screening; determining a second tumor mask identifying a second set of lesions present in a followup PET/CT scan captured at followup; identifying, in the second tumor mask, a first matching lesion that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the baseline PET/CT scan and the first matching lesion in the followup PET/CT scan, a change in metabolic level between screening and followup; and determining, based at least on the change in metabolic level between screening and followup, a response to a treatment for a disease.

In another aspect, there is provided a computer program product including a non-transitory computer readable medium. The non-transitory computer readable medium may store instructions, which when executed by at least one data processor, result in operations including: determining a first tumor mask identifying a first set of lesions present in a baseline positron emission tomography and computed tomography (PET/CT) scan captured at screening; determining a second tumor mask identifying a second set of lesions present in a followup PET/CT scan captured at followup; identifying, in the second tumor mask, a first matching lesion that corresponds to a first target lesion selected from the first set of lesions in the first tumor mask, the first matching lesion being identified based at least on an overlap between a first plurality of pixel coordinates of the first target lesion in the first tumor mask and a second plurality of aligned pixel coordinates of the first matching lesion in the second tumor mask aligned with the first tumor mask; determining, based on at least a metabolic level of each of the first target lesion in the baseline PET/CT scan and the first matching lesion in the followup PET/CT scan, a change in metabolic level between screening and followup; and determining, based at least on the change in metabolic level between screening and followup, a response to a treatment for a disease.

In some variations, one or more features disclosed herein including the following features can optionally be included in any feasible combination.

In some variations, a segmentation model may be applied to generate each of the first tumor mask and the second tumor mask.

In some variations, the segmentation model may include a longitudinal segmentation model trained to generate each of the first tumor mask and the second tumor mask based on the first PET/CT scan from the first timepoint and the second PET/CT scan from the second timepoint.

In some variations, a first CT scan in the first PET/CT scan and a second CT scan in the second PET/CT scan may be registered by at least determining one or more transformations to align at least a first fiducial marker in the first PET/CT scan with a second fiducial marker in the second PET/CT scan; and in response to determining that the first tumor mask includes at least the first target lesion and the second tumor mask includes at least the first matching lesion, applying the one or more transformations to a third plurality of unaligned pixel coordinates of the first matching lesion in the second tumor mask in order to generate the second plurality of aligned pixels coordinates of the first matching lesion in the second tumor mask aligned with the first tumor mask.

In some variations, the overlap between the first plurality of pixel coordinates of the first target lesion and the second plurality of aligned pixel coordinates of the first matching lesion may be determined by at least determining an intersection-over-union (IOU) between the first plurality of pixel coordinates and the second plurality of aligned pixel coordinates.

In some variations, the first matching lesion from the second timepoint may be identified as corresponding to the first target lesion from the first timepoint based at least on the intersection-over-union (IOU) satisfying one or more thresholds.

In some variations, in response to determining that the first tumor mask includes at least the first target lesion but the second tumor mask fails to include any lesions, the response to the treatment for the disease may be determined as complete metabolic response (CMR).

In some variations, in response to determining that the first tumor mask fails to include any lesions and the second tumor mask includes at least the first matching lesion, the response to the treatment for the disease may be determined as progressive metabolic disease (PMD).

In some variations, a matching lesion for every target lesion selected from the first set of lesions may be identified based at least on the first tumor mask and the second tumor mask. The identifying may include determining, for each target lesion selected from the first set of lesions, an overlap with each lesion in the second set of lesions.

In some variations, at least one new lesion in the second set of lesions that fail to match any target selections selected from the first set of lesions may be identified based at least on the first tumor mask and the second tumor mask. In response to identifying the at least one new lesions in the second set of lesions, the response to the treatment may be determined as progressive metabolic disease (PMD).

In some variations, a first overall metabolic level of the first set of lesions at the first timepoint may be determined based at least on a first metabolic level of the first target lesion. A second overall metabolic level of the second set of lesions at the second timepoint may be determined based at least on a second metabolic level of the first matching lesion. The change in metabolic level between the first timepoint and the second timepoint may be determined based at least on a difference between the first overall metabolic level at the first timepoint and the second overall metabolic level at the second timepoint.

In some variations, each of the first overall metabolic level at the first timepoint and the second overall metabolic level at the second timepoint may include a mean, a median, a maximum, a minimum, a mode, and/or a range of an individual metabolic level of each constituent lesion in a corresponding set of lesions.

In some variations, each of the first overall metabolic level at the first timepoint and the second overall metabolic level at the second timepoint may include an average of an individual metabolic level of each constituent lesion in a corresponding set of lesions weighted by a volume of each constituent lesion.

In some variations, the metabolic level of each of the first target lesion and the first matching lesion may be determined based at least on a metabolic level of each constituent pixel.

In some variations, the metabolic level of each of the first target lesion and the first matching lesion may be determined based at least on a mean, a median, a mode, a maximum, a minimum, and/or a range across the metabolic level of each constituent pixel.

In some variations, the metabolic level of each constituent pixel may correspond to an intensity of each constituent pixel in a corresponding PET scan.

In some variations, the response to the treatment may be determined as progressive metabolic disease (PMD) based at least on the change in metabolic level satisfying a first threshold.

In some variations, the response to the treatment may be determined as stable metabolic disease (SMD) based at least on the change in metabolic level satisfying a second threshold but failing to satisfy the first threshold.

In some variations, the response to the treatment may be determined as partial metabolic response (PMR) based at least on the change in metabolic level failing to satisfy the first threshold and the second threshold.

In some variations, the response to the treatment for the disease is determined by at least determining, based at least on the change in metabolic level between the first timepoint and the second timepoint satisfying a first threshold, that a metabolic level exhibits a threshold increase between the first timepoint and the second timepoint; and in response to determining that the metabolic level exhibits the threshold increase, determining that the response to the treatment for the disease is progressive metabolic disease (PMD).

In some variations, the response to the treatment for the disease may be further determined by at least determining, based at least on the change in metabolic level between the first timepoint and the second timepoint satisfying a second threshold, that the metabolic level exhibits a threshold decrease between the first timepoint and the second timepoint; and in response to determining that the metabolic level exhibits the threshold decrease, determining that the response to the treatment for the disease is partial metabolic response (PMR).

In some variations, the response to the treatment for the disease may be further determined by at least determining, based at least on the change in metabolic level between the first timepoint and the second timepoint failing to satisfy the first threshold and the second threshold, that the metabolic level fails to exhibit the threshold increase and the threshold decrease between the first timepoint; and in response to determining that the metabolic level fails to exhibit the threshold increase and the threshold decrease, determining that the response to the treatment for the disease is stable metabolic disease (SMD).

In some variations, at least one of a metabolic level and a size of each lesion in the first set of lesions depicted in the first PET/CT scan may be determined based at least on the first tumor mask. The first target lesion may be selected for inclusion in a set of target lesions based on the at least one of the metabolic level and the size of each lesion in the first set of lesions.

In some variations, the first target lesion may be selected for inclusion in the set of target lesions based at least on the metabolic level and/or the size of the first target lesion satisfying one or more thresholds.

In some variations, the first target lesion may be selected for inclusion in the set of target lesions based at least on the first target lesion being one of a threshold quantity of lesions having a highest metabolic level and/or a largest size within the first set of lesions.

In some variations, the first PET/CT scan may be performed prior to the treatment of the disease and the second PET/CT scan may be performed subsequent to the treatment for the disease.

In some variations, the first PET/CT scan may be a baseline scan performed at screening or upon a relapse of the disease and the second PET/CT scan may be a follow-up scan performed after the first PET/CT scan.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to assessment, staging, and response criteria for lymphomas (e.g., Hodgkin lymphoma, non-Hodgkin lymphoma (NHL), and/or the like), such as the European Organization for Research and Treatment of Cancer (EORTC) criteria, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a machine learning based medical imaging analysis system, in accordance with some example embodiments;

FIG. 2 depicts a schematic diagram illustrating an example of a process for machine learning enabled determination of disease progression and treatment response using medical imaging data, in accordance with some example embodiments;

FIG. 3 depicts a schematic diagram illustrating an example aligning two sets of pixels (or voxels) and determining the intersection-over-union (IOU) therebetween, in accordance with some example embodiments;

FIG. 4 depicts a flowchart illustrating an example of a process for machine learning enabled determination of disease progress and treatment response based on medical imaging data, in accordance with some example embodiments;

FIG. 5 depicts a flowchart illustrating an example of a process for tumor matching, in accordance with some example embodiments;

FIG. 6 depicts a flowchart illustrating an example of a process for determination of treatment response, in accordance with some example embodiments;

FIG. 7 depicts a table illustrating the accuracy of machine learning enabled determination of disease progress and treatment response using medical imaging data from breast cancer patients, in accordance with some embodiments;

FIG. 8 depicts a graph illustrating the accuracy of machine learning enabled determination of disease progress and treatment response using medical imaging data, in accordance with some example embodiments;

FIG. 9 depicts a block diagram illustrating an example of a computing system, in accordance with some example embodiments.

When practical, similar reference numbers denote similar structures, features, or elements.

### DETAILED DESCRIPTION

Various modalities of medical imaging may be applied to obtain data characterizing a subject's internal anatomy as well as pathophysiology. The resulting medical imaging data may have a variety of applications including, for example, disease diagnosis, prognosis, treatment planning, interoperative navigation, post operative monitoring, research, and/or the like. Computed tomography (CT) is one example of a three-dimensional imaging modality in which a series of X-rays are captured to create cross-sectional images (e.g., patches, slices, and/or the like) of the bones, blood vessels, and soft tissues inside the body. A computed tomography scan may be a three-dimensional volume formed by a series of two-dimensional images in which each pixel (or voxel) is associated with an intensity value indicative of a tissue density or x-ray attenuation at the corresponding location in the subject's body (e.g., the section thickness). Another example of a three-dimensional imaging modality is positron emission tomography (PET), which captures radioactivity signals indicative of cellular metabolic activities inside the subject's body. A positron emission tomography scan may also be a three-dimensional volume formed by a series of two-dimension images in which each pixel (or voxel) is associated with an intensity value indicative of the level of cellular metabolic activity (e.g., glucose uptake) at the corresponding location in the subject's body. In some cases, a single gantry incorporating a positron emission tomography (PET) scanner and a computed tomography (CT) scanner may be capable of acquiring positron emission tomography (PET) scans and computed tomography (CT) scans during a same session. The resulting positron emission tomography (PET) scan and computed tomography (CT) scan may be combined into a single superposed (e.g., transformed using a transformation matrix) image (e.g., a PET-CT scan) in which the spatial distribution of metabolic activities depicted in the positron emission tomography (PET) scan is aligned with the anatomical structures depicted in the computed tomography (CT) scan.

In some example embodiments, an analysis controller may determine, based at least on a first PET-CT scan from a first timepoint and a second PET-CT scan from a second timepoint, a treatment response for a patient associated with the first PET-CT scan and the second PET-CT scan. For example, in some cases, the analysis controller may determine, based at least on a change in metabolic level exhibited by one or more lesions that are present in both the first PET-CT scan and the second PET-CT scan, the treatment response. In some cases, the first PET-CT scan from the first timepoint may be a baseline scan captured during an initial patient screening or upon relapse of the disease (e.g., to rebaseline the patient) while the second PET-CT scan from the second timepoint may be a follow-up scan captured during a subsequent patient followup. Accordingly, in some cases, the analysis controller may determine, based at least on the change in metabolic level that occur between initial patient screening (or rebaselining) and follow-up, the treatment response. For instance, in some cases, the analysis controller may determine, based at least on the change in metabolic level satisfying one or more thresholds, the treatment response as progressive metabolic disease (PMD), stable metabolic disease (SMD), partial metabolic response (PMR), or complete metabolic response (CMR).

In some example embodiments, the analysis controller may determine the treatment response based on the change in metabolic level exhibited by one or more existing lesions from the first PET-CT scan that are also present in the second PET-CT scan. For example, in some cases, the analysis controller may select, based at least on a first tumor mask identifying a first set of lesions present in the first PET-CT scan, one or more target lesions in the first PET-CT scan. Furthermore, the analysis controller may identify, based at least on the first tumor mask and a second tumor mask identifying a second set of lesions present in the second PET-CT scan, one or more matching lesions in the second PET-CT scan that correspond to the one or more target lesions in the first PET-CT scan. In some cases, the change in metabolic level between the first timepoint (e.g., screening or rebaselining) and the second timepoint (e.g., followup) may be determined based at least on the change in metabolic level exhibited by the one or more target lesions in the first PET-CT scan and the one or more matching lesions in the second PET-CT scan. It should be appreciated that an existing lesion, or a lesion that is present at both the first timepoint and the second timepoint, may have a different clinical significance than disappearing lesions (or lesions that are present at the first timepoint but not the second timepoint) and new lesions (or lesions that are present at the second timepoint but not the first timepoint). As such, existing lesions should be differentiated from new lesions and disappearing lesions. Moreover, to correctly determine how the metabolic activities of a lesion changes between the first timepoint and the second timepoint, it may be crucial to correctly identify the same lesions in the first PET/CT scan from the first timepoint and in the second PET/CT scan from the second timepoint. As described in more details below, a matching lesion in the second PET/CT scan that is the same lesion as a target lesion in the first PET/CT may be identified based on an overlap between the two lesions in the corresponding lesion masks.

In some example embodiments, the analysis controller may generate each of the first tumor mask and the second tumor mask by at least applying a segmentation model to the corresponding PET-CT scan. For example, in some cases, the segmentation model may be a machine learning model trained classify each pixel (or voxel) within a PET-CT scan by at least assigning, to each pixel (or voxel), a first label (e.g., having a first value such as “1”) to indicate that the pixel (or voxel) is part of a lesion and a second label (e.g., having a second value such as “0”) to indicate that the pixel is not part of a lesion. In some cases, the segmentation model may be coupled with a longitudinal segmentation model trained to further refine each of the first tumor mask and the second tumor mask based on the first PET-CT scan as well as the second PET-CT scan. For instance, in some cases, the longitudinal segmentation model may ingest the first PET-CT scan, the first tumor mask, the second PET-CT scan, and the second tumor mask. Moreover, the longitudinal segmentation model may update each of the first tumor mask and the second tumor mask based on the first PET-CT scan and the second PET-CT scan. In doing so, the longitudinal segmentation model may refine each of the first tumor mask and the second tumor mask to reduce false positives in which a pixel (or voxel) that is not part of a lesion is identified as such as well as false negatives in which a pixel (or voxel) that is a part of a lesion is not identified as such.

In some example embodiments, the analysis controller may align the first tumor mask and the second tumor mask prior to identifying, within the second tumor mask, the one or more matching lesions that correspond to (or are the same lesions as) the one or more target lesions in the first tumor mask. Because the first PET-CT scan and the second PET-CT scans are captured at different times, sometimes by different technicians and/or using different scanners, the first PET/CT scan and the second PET/CT scan may be misaligned, meaning that a first pixel (or a first voxel) in the first PET/CT scan may depict a different part of the anatomy than a second pixel (or a second voxel) having the same coordinates (e.g., (x, y, z) coordinates) in the second PET/CT scan. This misalignment extends to the tumor masks generated therefrom, meaning that a first pixel (or first voxel) in the first tumor mask that depicts the same portion of a same lesion as a second pixel (or second voxel) in the second tumor mask may not necessarily be associated with the same coordinates. Accordingly, aligning the first tumor mask and the second tumor mask may include transforming (e.g., translating, rotating, scaling, and/or the like) the coordinates (e.g., (x, y, z) coordinates) of the pixels (or voxels) in the second CT scan such that the pixels (or voxels) depicting the same fiducial markers (e.g., anatomical features) in the first CT scan and the second CT scan are associated with the same coordinates. For example, in some cases, the analysis controller may align the first tumor mask and the second tumor mask by at least registering the first CT scan and the second CT scan based on one or more fiducial markers (e.g., anatomical landmarks) in the corresponding CT scans. Moreover, in some cases, the registration of the first CT scan and the second CT scan may include determining one or more transformations (e.g., affine transformations and/or the like) to align a first fiducial marker in the first PET/CT scan with a second fiducial marker in the second PET/CT scan. These transformations may be applied to the coordinates of the pixels (or voxels) in the second tumor mask to align the second tumor mask with the first tumor mask, thus generating an aligned second tumor mask whose coordinates are aligned with those of the first tumor mask. As described in more details below, the matching lesions in the second tumor mask may be identified based on the coordinates of the pixels (or voxels) of the one or more target lesions in the first tumor mask and the aligned coordinates of the pixels (or voxels) of the lesions in the second tumor mask.

In some example embodiments, the analysis controller may determine the treatment response (or disease progression) based on the change in metabolic level exhibited by a set of target lesions selected from the first set of lesions present in the first tumor mask. In some cases, the analysis controller may impose one or more criteria when selecting, from the first set of lesions present in the first tumor mask, one or more lesions for inclusion in the set of target lesions. For example, in some cases, the analysis controller may select, based at least on a metabolic level and/or a size (e.g., volume, diameter, and/or the like) of each lesion present in the first tumor mask, the one or more lesions for inclusion in the set of target lesions. In some cases, the analysis controller may select, for inclusion in the set of target lesions, a threshold quantity of the lesions present in the first tumor mask having a highest metabolic level and/or a largest size (e.g., volume, diameter, and/or the like). Alternatively and/or additionally, in some cases, the analysis controller may select, for inclusion in the set of target lesions, one or more of the lesions present in the first tumor mask that exhibit a threshold metabolic level and/or a threshold size (e.g., volume, diameter, and/or the like). In some cases, the selection of the set of target lesions may include generating a selected tumor mask in which each pixel (or voxel) depicting a target lesion is associated with a first label (e.g., having a first value such as “1”) and each pixel (or voxel) not depicting a target lesion is associated with a second label (e.g., having a second value such as “0”). As described in more detail below, the analysis controller may determine the treatment response (or disease progress) by at least on the change in metabolic level exhibited by the one or more lesions selected for inclusion in the set of target lesions.

In some example embodiments, the analysis controller may identify, based at least on the first tumor mask and the based at least on the first tumor mask (e.g., the selected tumor mask generated therefrom) and the aligned second tumor mask, one or more matching lesions in the aligned second tumor mask that correspond a target lesion first tumor mask. For example, in some cases, the analysis controller may determine, for each target lesion in the selected tumor mask, an overlap between the target lesion and every lesion present in the aligned second tumor mask. In some cases, the analysis controller may determine the overlap between a target lesion in the selected tumor mask and a lesion in the aligned second tumor mask by at least determining an intersection-over-union (IOU) between a first plurality of pixel coordinates of the target lesion in the selected tumor mask and a second plurality of pixel coordinates of in the lesion in the aligned second tumor mask. Where the intersection-over-union (IOU) satisfies one or more thresholds, the analysis controller may identify the lesion in the aligned second tumor mask as a matching lesion for the target lesion in the first tumor mask. In some cases, upon identifying the matching lesions in the aligned second tumor mask, the analysis controller may generate a matched tumor mask in which each pixel (or voxel) in the aligned second tumor mask depicting a matching lesion is associated with a first label (e.g., a first value such as “1”) and each pixel (or voxel) in the aligned second tumor mask not depicting a matching lesion is associated with a second label (e.g., a second value such as “0”).

In some example embodiments, the analysis controller may determine, based at least on the treatment response (or disease progression) based at least on a change in metabolic level between the first timepoint of the first PET/CT scan and the second timepoint of the second PET/CT scan. In some cases, the change in metabolic level between the first timepoint and the second timepoint may correspond to a difference between a first metabolic level of the target lesions at the first timepoint and a second metabolic level of the matching lesions at the second timepoint. Accordingly, in some cases, the analysis controller may determine the change in metabolic level by at least determining, based at least on the selected tumor mask and the corresponding first PET-CT scan, the first metabolic level of the target lesions at the first timepoint. Moreover, the analysis controller may determine the change in metabolic level by at least determining, based at least on the matched tumor mask and the corresponding second PET/CT scan, the second metabolic level of the matching lesions at the second timepoint. In some cases, the metabolic level at a particular timepoint may correspond to a mean, a median, a mode, a maximum, a minimum, and/or a range of the metabolic levels of the individual lesions present at that timepoint. Alternatively, in some cases, the metabolic level at a particular timepoint may correspond to an average of the metabolic levels of the individual lesions present at the timepoint weighted by the size of each lesion (e.g., diameter, volume and/or the like). It should be appreciated that in this context, metabolic level may be quantified by standard uptake value (SUV), which corresponds to the ratio of a first concentration of a radiopharmaceutical (e.g., fluorodeoxyglucose (FDG)) in a volume of tissue in microcuries of injected agent per volume to a second concentration in the body if uniformly distributed (e.g., as determined by a standard body phantom).

In some example embodiments, the analysis controller may determine that no matching lesions are present in the aligned second tumor mask, in which case the analysis controller may determine that the treatment response is complete metabolic response (CMR). For example, in some cases, the analysis controller may determine that no matching lesions are present in the aligned second tumor mask if the analysis controller is unable to identify, within the aligned second tumor mask, at least one lesion whose aligned pixel coordinates exhibit a threshold overlap (e.g., a threshold intersection-over-union (IOU)) with the pixel coordinates of the target lesions in the first tumor mask. Alternatively, the analysis controller may identify, within the aligned second tumor mask, one or more new lesions that are not present in the first tumor mask. For example, in some cases, the analysis controller may determine that a lesion in the aligned second tumor mask is a new lesion if that lesion fails to match any of the target lesions in the target tumor mask generated from the first tumor mask. In instances where the analysis controller detects the presence of one or more new lesions in the aligned second tumor mask, the analysis controller may determine that the treatment response is progressive metabolic disease (PMD). In some cases, the analysis controller may determine that the treatment response is progressive metabolic disease (PMD) if the one or more new lesions in the aligned second tumor mask exhibit a threshold metabolic level and/or a threshold size (e.g., volume, diameter, and/or the like).

In some example embodiments, the analysis controller may determine, based at least on the change in metabolic level between the first timepoint and the second timepoint satisfying one or more threshold, the treatment response as one of progressive metabolic disease (PMD), stable metabolic disease (SMD), partial metabolic response (PMR), or complete metabolic response (CMR). For example, in some cases, the analysis controller may determine that the change in metabolic level between the first timepoint and the second timepoint as exhibiting a threshold increase if the change in metabolic level satisfies a first threshold. Accordingly, the analysis controller may determine that the treatment response as progressive metabolic disease (PMD). Alternatively, in instances where the analysis controller determines that the change in metabolic level between the first timepoint and the second timepoint as exhibiting a threshold decrease based on the change in metabolic level satisfying a second threshold, the analysis controller may determine that the treatment response as partial metabolic response. In instances where the change in metabolic level between the first timepoint and the second timepoint fails to satisfy the first threshold and the second threshold, the analysis controller may determine that the treatment response is stable metabolic disease.

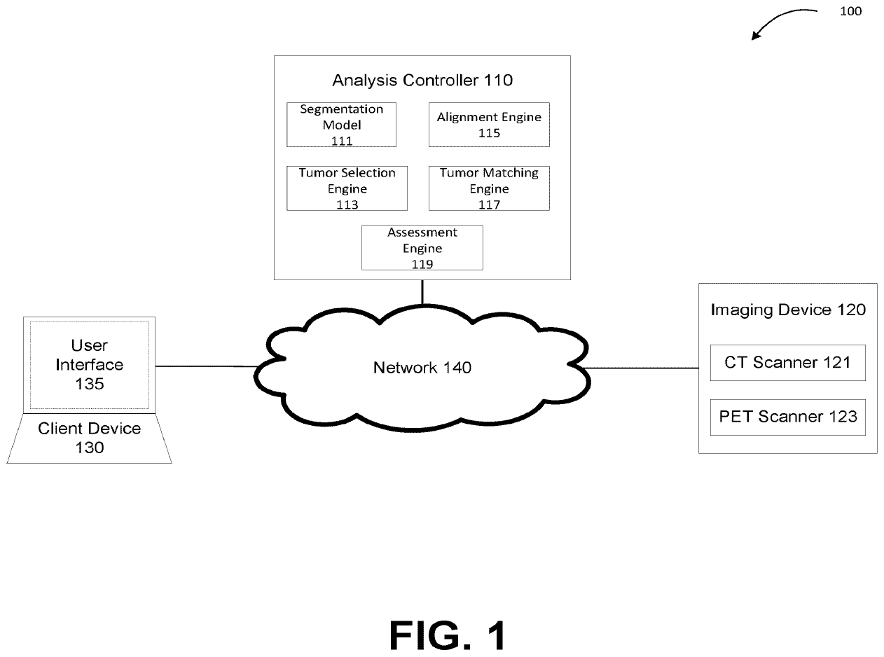

FIG. 1 depicts a system diagram illustrating an example of a machine learning based medical imaging analysis system 100, in accordance with some example embodiments. Referring to FIG. 1, the machine learning based medical imaging analysis system 100 may include an analysis controller 110, one or more imaging devices 120, and a client device 130. As shown in FIG. 1, the analysis controller 110, the one or more imaging devices 120, and the client device 130 may be communicatively coupled via a network 140. The one or more imaging devices 120 may include, for example, a computed tomography (CT) scanner 121 and a positron emission tomography (PET) scanner 123. The client device 130 may be a processor-based device including, for example, a smartphone, a tablet computer, a wearable apparatus, a virtual assistant, an Internet-of-Things (IoT) appliance, and/or the like. The network 140 may be a wired network and/or a wireless network including, for example, a wide area network (WAN), a local area network (LAN), a virtual local area network (VLAN), a public land mobile network (PLMN), the Internet, and/or the like.

In some example embodiments, the analysis controller 110 may determine, based at least on positron emission tomography (PET) scans and computed tomography (CT) scans generated by the one or more imaging devices 120 (e.g., the computed tomography scanner 121, the positron emission tomography scanner (PET) scanner 123, and/or the like), a treatment response or a disease progression. For example, in some cases, the analysis controller 110 may determine, based at least on a first PET-CT scan from a first timepoint and a second PET-CT scan from a second timepoint, a treatment response (or a disease progression) for a patient associated with the first PET-CT scan and the second PET-CT scan. In some cases, the first PET-CT scan from the first timepoint may be a baseline scan captured during an initial patient screening or upon relapse of the disease (e.g., to rebaseline the patient) while the second PET-CT scan from the second timepoint may be a follow-up scan captured during a subsequent patient follow-up. For instance, in some cases, the analysis controller 110 may determine, based at least on a change in metabolic level between the first timepoint and the second timepoint, the treatment response (or the disease progression). In particular, described in more details below, the analysis controller 110 may determine the treatment response (or disease progression) by at least determining the change in metabolic level exhibited by one or more lesions that are present in both the first PET-CT scan and the second PET-CT scan. Furthermore, as described in more details below, the analysis controller 110 may determine, based at least on the change in metabolic level of the one or more lesions satisfying one or more thresholds, the treatment response as progressive metabolic disease (PMD), stable metabolic disease (SMD), partial metabolic response (PMR), or complete metabolic response (CMR).

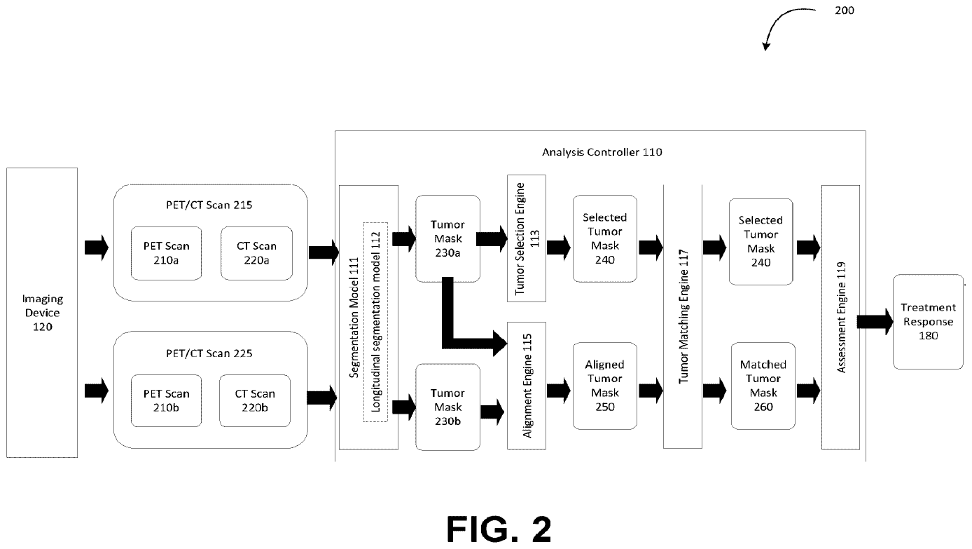

To further illustrate, FIG. 2 depicts a schematic diagram illustrating an example of a process 200 for machine learning enabled determination of treatment response, in accordance with some example embodiments. Referring to FIG. 2, the analysis controller 110 may receive, from the one or more imaging devices 120, a first positron emission tomography (PET) and computed tomography (CT) scan 215 from a first timepoint that includes a first PET scan 210a and a first CT scan 220a from the first timepoint. In some instances, the first PET/CT scan 215 from the first timepoint may be a baseline scan captured during an initial patient screening or upon relapse of a disease, for example, to rebaseline the patient. In some cases, the first PET/CT scan 215 may be captured prior to administering one or more treatments for a disease. As shown in FIG. 2, in some cases, the analysis controller 110 may also receive, from the one or more imaging devices 120, a second PET/CT scan 225 from a second timepoint that includes a second PET scan 210b and a second CT scan 220b from the second timepoint. In some instances, the second PET/CT scan 225 from the second timepoint may be a follow-up scan captured, for example, during a patient follow-up. In some instances, the second PET/CT scan 225 may be captured while the patient is undergoing treatment for the disease or after the completion of the treatment.

In some example embodiments, the analysis controller 110 may preprocess the first PET/CT scan 215 and the second PET/CT scan 225 including by segmenting each of the first PET/CT scan 215 and the second PET/CT scan 225. For example, the analysis controller 110 may include a segmentation model 111, which may be applied to segment the first PET/CT scan 215 to generate a first tumor mask 230a in which each pixel in the first PET/CT scan 215 depicting a lesion is assigned a first label (e.g., having a first value such as “1”) and each pixel in the first PET/CT scan 215 not depicting a lesion is assigned a second label (e.g., having a second value such as “0”). Moreover, as shown in FIG. 2, the segmentation model 111 may also segment the second PET/CT scan 225 to generate a second tumor mask 230b in which each pixel in the second PET/CT scan 225 depicting a lesion is assigned a first label (e.g., having a first value such as “1”) and each pixel in the second PET/CT scan 225 not depicting a lesion is assigned a second label (e.g., having a second value such as “0”).

In some example embodiments, the preprocessing of the first PET/CT scan 215 and the second PET/CT scan 225 may also include partitioning each of the first PET/CT scan 215 and the second PET/CT scan 225 into two or more regions. For example, in some cases, the segmentation model 111 may partition, based on one or more anatomical landmarks, the first positron emission tomography (PET) scan 210a, the first computed tomography (CT) scan 220a, the second positron emission tomography (PET) scan 210b, and the second computed tomography (CT) scan 220b into two or more anatomical regions. Examples of anatomical regions include a head and neck region, a chest region, and an abdomen and pelvis region.

In some example embodiments, the segmentation model 111 may be coupled with (or include) a longitudinal segmentation model 112 trained to update the first tumor mask 230a and the second tumor mask 230b based on the first PET/CT scan 215 and the second PET/CT scan 225. The quality of a PET/CT scan may be quantified by the signal-to-noise (SNR) ratio. In this context, the signal in the PET/CT scan may refer to the difference between the intensity of the pixels that depict a lesion and background pixels, which are those pixels not depicting a lesion. Meanwhile, the noise in the PET/CT scan may refer to the standard deviation of the intensities of the background pixels. When viewed as a whole, the background pixels in the PET/CT scan may exhibit a high standard deviation (or greater variation) in intensity at least because the PET/CT scan may span multiple anatomical regions. In those cases, the signal associated with the pixels depicting lesions may not be sufficiently high to enable accurate segmentation (or differentiation between background pixels and pixels depicting lesions) by the longitudinal segmentation model 112. Accordingly, in some cases, the performance of the longitudinal segmentation model 112 may be improved by the longitudinal segmentation model 112 operating on portions (or patches) of the first PET/CT scan 215 and the second PET/CT scan 225 depicting one or more lesions instead of the first PET/CT scan 215 and the second PET/CT scan 225 in their entirety. For example, in some cases, a first patch may be extracted from the first PET/CT scan 215 to include a first lesion associated with the first tumor mask 230a while a second patch may be extracted from the second PET/CT scan 225 include a second lesion associated with the second tumor mask 230b. In this context, the first patch may include a subset of pixels from the first PET/CT scan 215 that depict the first lesion while the second patch may be include a subset of pixels from the second PET/CT scan 225 that depict the second lesion.

In some example embodiments, the longitudinal segmentation model 112 may update, based at least on the first patch and the second patch, the first tumor mask 230a and the second tumor mask 230b. For instance, the longitudinal segmentation model 112 may determine whether a pixel is a part of a lesion based on the level of metabolic activity and the tissue density (or x-ray attenuation) exhibited by the pixel and one or more neighboring pixels across the first timepoint and the second timepoint. In doing so, the longitudinal segmentation model 112 may update the first tumor mask 230a and/or the second tumor mask 230b including by updating the label assigned to one or more pixels in the first tumor mask 230a and/or the second tumor mask 230b. Accordingly, in some cases, a pixel previously classified (e.g., by the segmentation model 111) as being a part of a lesion is reclassified by the longitudinal segmentation model 112 as not being part of a lesion and a pixel previously classified (e.g., by the segmentation model 11) as not being a part of a lesion is reclassified by the longitudinal segmentation model as being a part of a lesion. As noted, the longitudinal segmentation model 112 may ingest the first patch and the second patch instead of the first PET/CT scan 215 and the second PET/CT scan 225 in their entirety in order to increase the signal-to-noise (SNR) associated with the pixels depicting the first lesion and the second lesion.

In some example embodiments, the analysis controller 110 may continue the process 200, for example, with the selection of target lesions and the alignment of the first tumor mask 230a and the second tumor mask 230b if each of the first tumor mask 230a and the second tumor mask 230b includes at least one lesion. The analysis controller 110 may terminate further processing of the first PET/CT scan 215 and the second PET/CT scan 225 in cases where the first tumor mask 230a or the second tumor mask 230b fail to contain at least one lesion. For example, in instances where the first tumor mask 230a fails to contain at least one lesion but one or more lesions are present in the second tumor mask 230b, the assessment engine 119 may determine that the one or more lesions in the second tumor mask 230b are new lesions and that the treatment response 180 is therefore progressive metabolic disease (PMD). In instances where the first tumor mask 230a includes one or more lesions but the second tumor mask 230b fails to include at least one lesion, the assessment engine 119 may determine that the treatment response 180 is complete metabolic response (CMR). In cases where no lesions are present in both the first tumor mask 230a and the second tumor mask 230b, the analysis controller 110 may determine the occurrence of certain irregularities, for example, during the segmentation of the first PET/CT scan 215 and the second PET/CT scan 225 and return a corresponding error message. It should be appreciated that the termination of further processing based on the absence of at least one lesion in the first tumor mask 230a and/or the second tumor mask 230b may obviate the computational burden associated therewith.

Referring again to FIG. 2, in some example embodiments, the analysis controller 110 may include a tumor selection engine 113 that selects, for inclusion in a set of target lesions, one or more lesions present in the first tumor mask 230a. For example, in some cases, the tumor selection engine 113 may select, based at least on a metabolic level and/or a size (e.g., volume, diameter, and/or the like) of each lesion present in the first tumor mask 230a, one or more lesions for inclusion in the set of target lesions. In some cases, the tumor selection engine 113 may select, from the first tumor mask 230a, a threshold quantity of lesions (e.g., 5 lesions) having a highest metabolic level and/or a largest size (e.g., volume, diameter, and/or the like). Alternatively and/or additionally, the tumor selection engine 113 may select, from the first tumor mask, one or more lesions whose metabolic level and/or size (e.g., volume, diameter, and/or the like) satisfy one or more thresholds. In some cases, the tumor selection engine 113 may generate a selected tumor mask 240 in which each pixel depicting a selected lesion is assigned a first label (e.g., having a first value such as “1”) and each pixel not depicting a selected lesion is assigned a second label (e.g., having a second value such as “0”).

As noted, each pixel in a positron emission tomography (PET) scan may be associated with an intensity value corresponding to a level of metabolic activity (e.g., standard uptake value (SUV)). Accordingly, it should be appreciated that in this context, the metabolic level of a lesion may be determined based on the standard uptake value (SUV) of one or more pixels (or voxels) in a PET scan identified by the corresponding tumor mask as depicting the lesion. Because the lesion may encompass multiple pixels (or voxels) in the PET scan, the metabolic level of the lesion may correspond to one or more of a mean, a median, a mode, a maximum, a minimum, or a range of the standard uptake value (SUV) across the pixels (or voxels). Meanwhile, in some cases, the size of a lesion (e.g., the volume, diameter, and/or the like) may be determined based on the quantity of pixels (or voxels) identified, for example, by the corresponding tumor mask, as depicting the lesion. In some cases, the tumor selection engine 113 may select, for inclusion in the set of target lesions, one or more lesions based on the metabolic level of each lesion in the first tumor mask 230a weighted by the corresponding size (e.g., volume, diameter, and/or the like).

Referring again to FIG. 2, in some example embodiments, an alignment engine 115 may align the second tumor mask 230b with the first tumor mask 230a in order to identify, within the second tumor mask 230b, one or more matching lesions that correspond to the lesions in the first tumor mask 230a that have been selected for inclusion in the set of target lesions. For example, the alignment engine 115 may align the first tumor mask 230a and the second tumor mask 230b by least registering the first CT scan 220a and the second CT scan 220b, which includes determining one or more transformations to align a first fiducial marker (e.g., a first anatomical landmark) in the first CT scan 220a with a second fiducial marker (e.g., a second anatomical landmark) in the second CT scan 220b. As noted, in some cases, the selected tumor mask 240 may identify the lesions in the first tumor mask 230a selected for inclusion, for example, by the tumor selection engine 113, in the set of target lesions. Moreover, the registration may be performed based on the first CT scan 220a and the second CT scan 220b at least because the first PET scan 210a and the second PET scan 210b lack sufficiently clear fiducial markers (e.g., anatomical landmarks). In some cases, the one or more transformations may include affine transformations such as, for example, translations, rotations, scaling, and/or the like. When applied to the unaligned pixel coordinates in the second CT scan 220b, the one or more transformation may transform the unaligned pixel coordinates in the second CT scan 220b such that the resulting aligned pixel coordinates of the second fiducial marker (e.g., the second anatomical landmark) in the second CT scan 220b are match the pixel coordinates of the first fiducial marker (e.g., the first anatomical landmark) in the first CT scan 220a.

In some instances, the one or more transformations may be in the form of a transformation matrix (e.g., affine transformation matrix) that can be applied to the second tumor mask 230b in order to align the second tumor mask 230b with the first tumor mask 230a. As shown in FIG. 2, the alignment engine 115 may apply, to one or more pixel coordinates in the second tumor mask 230b, the one or more transformations (e.g., the transformation matrix) to generate an aligned tumor mask 250 having one or more aligned pixel coordinates. In some cases, the one or more transformations may be applied to a portion of the second tumor mask 230b or the second tumor mask 230b in its entirety.

Referring again to FIG. 2, the analysis controller 110 may include a tumor matching engine 117 that identifies, within the second tumor mask 230b aligned with the first tumor mask 230a (e.g., the aligned tumor mask 250), one or more matching lesions that corresponds to the target lesions selected from the first tumor mask 230a. For example, in instances where the selected tumor mask 240 identifies the set of target lesions from the first tumor mask 230a, the tumor matching lesions 117 may determine, for each lesion present in the selected tumor mask 240, a matching lesion in the second tumor mask 230b aligned with the first tumor mask 230a (e.g., the aligned tumor mask 250). In some cases, the one or more matching lesions may be identified by aligned tumor mask and matched tumor mask 260 in which pixels depicting a matching lesion are assigned a first label (e.g., having a first value such as “1”) and pixels not depicting a matching lesion are assigned a second label (e.g., having a second value such as “0”). As described in more details below, the assessment engine 180 may determine the treatment response 180 based on the change in metabolic level exhibited by the target lesions in the first PET/CT scan 215 and the matching lesions in the second PET/CT scan 225.

As noted, the tumor matching engine 117 may identify, for each target lesions in the set of target lesions selected from the first tumor mask 230a, a matching lesion present in the second tumor mask 230b. In some cases, the tumor matching engine 117 may determine whether a lesion in the second tumor mask 230b is a matching lesion that corresponds to a target lesion in the first tumor mask 230a by at least determining an overlap between a first plurality of pixel coordinates of the target lesion and a second plurality of aligned pixel coordinates of the lesion in the second tumor mask 230b aligned with the first tumor mask 230a (e.g., in the aligned tumor mask 250). In some cases, the aforementioned overlap may correspond to an intersection-over-union (IOU) between the first plurality of pixel coordinates of the target lesion and second plurality of aligned pixel coordinates of the lesion. For example, in some cases, the intersection-over-union (IOU) between two sets of pixels (or voxels), such as a first set of pixels depicting the target lesion in the first tumor mask 230a and a second set of pixels depicting the lesion in the second tumor mask 230b aligned with the first tumor mask 230a (e.g., the aligned tumor mask 250), may correspond to a ratio between a first quantity of pixels (or voxels) in the intersection between the two sets of pixels and a second quantity of pixels (or voxels) in the union between the two sets of pixels. In some cases, the intersection-over-union between the two sets of pixels (or voxels) may be determined based on the coordinates (e.g., (x, y, z) coordinates) of the constituent pixels (or voxels). In some cases, the tumor matching engine 117 may determine that the lesion in the second tumor mask 230b aligned with the first tumor mask 230a (e.g., the aligned tumor mask 250) is a matching lesion corresponding to the target lesion in the first tumor mask 230a (e.g., the selected tumor mask 240) if the intersection-over-union (IOU) between the corresponding sets of pixels satisfies one or more thresholds. In instances where the lesion in the second tumor mask 230b is determined to be a matching lesion corresponding to the target lesion in the first tumor mask 230a, the tumor matching engine 117 may generate the matched tumor mask 260 to include the matching lesion. Alternatively, in instances where the lesion in the second tumor mask 230b is not a matching lesion corresponding to the target lesion in the first tumor mask 230a (e.g., due to the intersection-over-union (IOU) therebetween failing to satisfy the one or more thresholds), the tumor matching engine 117 may determine whether one or more other lesions in the second tumor mask 230b aligned with the first tumor mask 230a (e.g., the aligned tumor mask 250) is a matching tumor. It should be appreciated that the tumor matching process may be iterative, meaning that tumor matching engine 117 may attempt to match each target lesion in the first tumor mask 230a (e.g., the selected tumor mask 240) to each lesion in the second tumor mask 230b aligned with the first tumor mask 230a (e.g., the aligned tumor mask 250) until either (i) a matching lesion having a threshold overlap is identified or (ii) the tumor matching engine 117 has examined every lesion in the second tumor mask 230b.

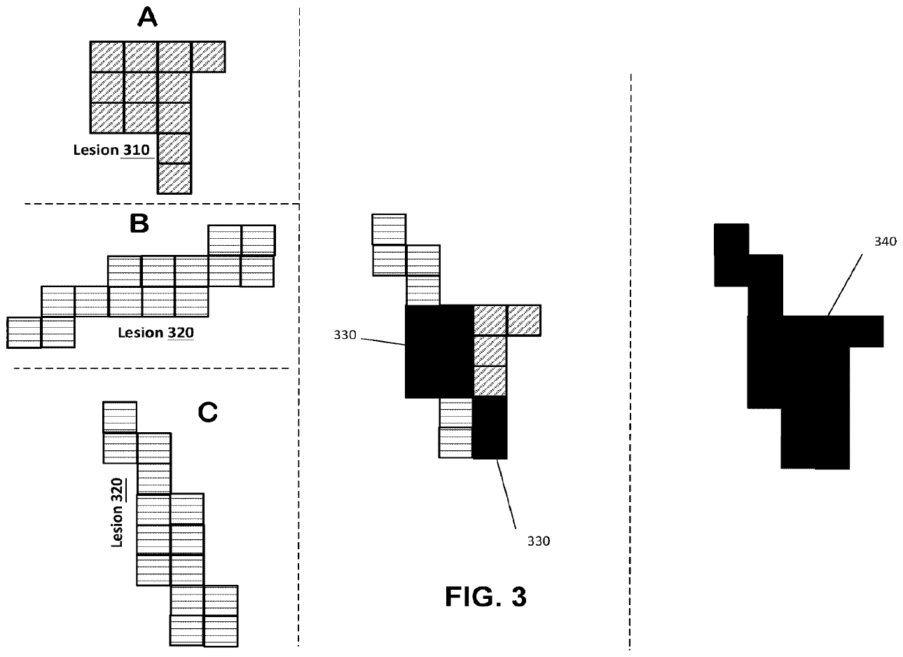

To further illustrate, FIG. 3 depicts a schematic diagram illustrating an example of aligning two sets of pixels (or voxels) and determining the intersection-over-union (IOU) therebetween, in accordance with some example embodiments. FIG. 3 shows pixel set A depicting a first lesion 310 and pixel set B depicting a second lesion 320. In some cases, the first lesion 310 may be a target lesion from the first tumor mask 230a and the second lesion 320 may be a lesion from the second tumor mask 230b. Moreover, in some cases, the coordinates of pixels (or voxels) in pixel set A may be with the coordinates of those in pixel set B in order to determine whether the second lesion 320 is a matching lesion that corresponds to the first lesion 310. Although FIG. 3 shows alignment and intersection-over-union computation for pixels in two-dimensional space, it should be appreciated that the same techniques may be applied to pixels (or voxels) in three-dimensional space.

Referring again to FIG. 3, in some cases, the coordinates of the pixels (or voxels) in pixel set B may not be aligned with the coordinates of the pixels in pixel set A. This misalignment, which may be attributed to differences in the technicians and/or scanners capturing the corresponding PET/CT scans, may give rise to false negatives during tumor matching where the second lesion 320 fails to match the first lesion 310 due to misalignment and not due to insufficient overlap (e.g., the intersection-over-union (IOU)) between the two lesions. As such, FIG. 3 shows that one or more transformations (e.g., affine transformations) may be applied to the coordinates of the pixels (or voxels) in pixel set B in order to generate pixel set C, which contains pixels (or voxels) whose coordinates are aligned with the coordinates of the pixels (or voxels) in pixel set A. For example, in some cases, the coordinates of the pixels (or voxels) in pixel set B may be rotated, translated, and/or scaled in order to generate the coordinates of the pixels (or voxels) in pixel set C. In the particular example shown in FIG. 3, the coordinates of the pixels (or voxels) in pixel set B was rotated (e.g., 90° clockwise) in order to generate the coordinates of the pixels (or voxels) in pixel set C. In some cases, these transformations may be applied to align a first fiducial marker (e.g., a first anatomical landmark) in a first CT scan associated with pixel set A and a second fiducial marker (e.g., a second anatomical landmark) in a second CT scan associated with pixel set B.

As shown in FIG. 3, in some cases, the tumor matching engine 117 may determine whether the second lesion 320 is a match for the first lesion 310 by at least determining an intersection-over-union (IOU) between pixel set A and pixel set C. As shown in FIG. 3, the intersection-over-union (IOU) between pixel set A and pixel set C may correspond to a ratio between a first quantity of pixels (or voxels) in the intersection 330 between pixel set A and pixel set C and a second quantity of pixels (or voxels) in the union 340 between pixel set A and pixel set C. As shown in FIG. 3, the intersection 330 between pixel set A and pixel set C may include one or more pixels that are in both pixel set A and pixel set C. Meanwhile, the union 340 there between may include one or more pixels that are in (i) both pixel set A and pixel set C, (ii) in pixel set A but not pixel set C, and (iii) in pixel set C but not in pixel set A. In some cases, the intersection 330 and the union 340 between pixel set A and pixel set C may be determined based on the coordinates (e.g., (x, y, z) coordinates) of the constituent pixels (or voxels). In this example, the intersection 330 between pixel set A and pixel set C includes eight pixels while the union 340 between pixel set A and pixel set C includes eighteen pixels. Accordingly, for the example shown in FIG. 3, the intersection-over-union between pixel set A and pixel set C is 0.44. In instances where that value satisfies one or more thresholds, then the tumor matching engine 117 may identify the second lesion 320 as a matching lesion for the first lesion 310.

Referring again to FIG. 2, the assessment engine 119, which may be a part of the analysis controller 110, may determine, based at least on a change in metabolic level between the target tumors selected from the first tumor mask 230a and the matching tumors in the second tumor mask 230b, the treatment response 180. For example, as noted, the treatment response 180 may be complete metabolic response (CMR) in instances where the first tumor mask 230a includes at least one target lesion but no lesions are present in the second tumor mask 230b. Alternatively, the treatment response 180 may be progressive metabolic disease (PMD) if no target lesions are present in the first tumor mask 230a but at least one lesion is present in the second tumor mask 230b. In some cases where the second tumor mask 230b may include one or more new lesions, which may be identified as such because these lesions fail to match any of the target lesions in the first tumor mask 230a, the treatment response 180 may be progressive metabolic disease (PMD) if these new lesions exhibit a threshold metabolic level and/or a threshold size (e.g., volume, diameter, and/or the like). In cases where the second tumor mask 230b includes at least one matching lesion corresponding to a target lesion in the first tumor mask 230a but no new lesions, the assessment engine 119 may determine, based at least on whether a difference between a first metabolic level at the first timepoint of the first tumor mask 230a and a second metabolic level of the second timepoint of the second tumor mask 230b satisfies one or more thresholds, the treatment response 180. For instance, the treatment response 180 may be determined as progressive metabolic disease (PMD) if the difference between the first metabolic level at the first timepoint and the second metabolic level at the second timepoint satisfies a first threshold. In cases where the difference between the first metabolic level at the first timepoint and the second metabolic level at the second timepoint satisfies a second threshold but not the first threshold, the assessment engine 119 may determine the treatment response 180 to be stable metabolic disease (SMD). If the difference between the first metabolic level at the first timepoint and the second metabolic level at the second timepoint fails to satisfy the first threshold and the second threshold, the assessment engine 119 may determine that the treatment response 180 is partial metabolic response (PMR).

In some example embodiments, the one or more thresholds may be set to differentiate between the presence (or absence) of a threshold increase in metabolic levels or a threshold decrease in metabolic levels. For example, the assessment engine 119 may detect an increase in metabolic levels between the first timepoint and the second timepoint if the difference between the first metabolic level at the first timepoint and the second metabolic level at the second timepoint satisfies a first threshold. If the difference between the first metabolic level at the first timepoint and the second metabolic level at the second timepoint further satisfies a second threshold, the assessment engine 110 may detect a threshold increase in metabolic levels, in which case the treatment response 180 may be determined to be progressive metabolic disease (PMD). If the difference between the first metabolic level at the first timepoint and the second metabolic level at the second timepoint fails to also satisfy the second threshold, the increase in metabolic levels may not reach a threshold increase and the treatment response 180 is therefore stable metabolic disease (SMD).

Alternatively, the assessment engine 119 may detect a decrease in metabolic levels between the first timepoint and the second timepoint if the difference between the first metabolic level at the first timepoint and the second metabolic level at the second timepoint fails to satisfy the first threshold. In some cases, if the difference between the first metabolic level at the first timepoint and the second metabolic level at the second timepoint further satisfies a second threshold, the assessment engine 110 may detect a threshold decrease in metabolic levels, in which case the treatment response 180 may be determined to be partial metabolic response (PMR). If the difference between the first metabolic level at the first timepoint and the second metabolic level at the second timepoint fails to also satisfy the second threshold, the decrease in metabolic levels may not reach a threshold decrease and the treatment response 180 is therefore stable metabolic disease (SMD).

In some example embodiments, the assessment engine 119 may determine the metabolic level for each timepoint based on the matched tumor mask 260 and the corresponding positron emission tomography (PET) scan. For example, as noted, the selected tumor mask 240, which may be generated from the first tumor mask 230a, includes pixels (or voxels) depicting a target lesion selected from the first tumor mask 230a being assigned a first label (e.g., having a first value such as “1”) and pixels (or voxels) not depicting a target lesion selected from the first tumor mask 230a being assigned a second label (e.g., having a second value such as “0”). Accordingly, in some cases, those pixels (or voxels) that depict target lesions in the first PET scan 210a may be identified based on the selected tumor mask 240. For instance, the pixels (or voxels) in the first PET scan 210a that depict target lesions may have the same coordinates (e.g., (x, y, z) coordinates) as the pixels (or voxels) in the selected tumor mask 240 assigned the first label (e.g., having the first value such as “1”).

In some cases, the intensity value of an individual pixel (or voxel) in the first PET scan 210a may correspond to a metabolic level (e.g., a standardized uptake value (SUV)) at the corresponding location in the subject's body. Accordingly, the first metabolic level at the first timepoint may be determined based on the intensity values of the pixels (or voxels) in the first PET scan 210a depicting one or more target lesions. In some cases, each target lesion in the selected tumor mask 240 may be associated with a metabolic level corresponding to a mean, a median, a mode, a maximum, a minimum, or a range of the intensity values of the pixels (or voxels) depicting the target lesion in the first PET scan 210a. For example, if a target lesion includes a first pixel and a second pixel in the first PET scan 210a, the metabolic level of that target lesion may be a mean, a median, a mode, a maximum, a minimum, or a range of a first intensity value of the first pixel and a second intensity value of the second pixel. Meanwhile, the first metabolic level may, in some cases, correspond to a mean, a median, a mode, a maximum, a minimum, or a range of the metabolic levels of every target lesion in the selected tumor mask 240. For instance, if the first PET scan 210a includes a first target lesion and a second target lesion, the first metabolic level may be a mean, a median, a mode, a maximum, a minimum, or a range of a first metabolic level of the first target lesion and a second metabolic level of the second target lesion. Alternatively, the first metabolic level may correspond to an average metabolic level across every target lesion in the selected tumor mask 240 weighted by the size (e.g., volume, diameter, and/or the like) of the target lesion. In the previous example in which the first PET scan 210a includes the first target lesion and the second target lesion, the first metabolic level may be an average of the first metabolic level of the first target lesion weighted by a first size of the first target lesion and the second metabolic level of the second target lesion weighted by a second size of the second target lesion.

In some example embodiments, the second metabolic level at the second timepoint may be determined based on the metabolic levels of the matching lesions present in the second tumor mask 230b. For example, in some cases, the matching lesions present in the second tumor mask 230b may be identified by the matched tumor mask 260 in which each pixel depicting a matching lesion is assigned a first label (e.g., having a first value such as “1”) and each pixel not depicting a matching lesion is assigned a second label (e.g., having a second value such as “0”). Accordingly, in some cases, pixels (or voxels) in the second PET scan 210b that depict matching lesions may have the same coordinates (e.g., (x, y, z) coordinates) as the pixels (or voxels) in the matched tumor mask 260 assigned the first label (e.g., having the first value such as “1”). Moreover, the second metabolic level at the second timepoint may be determined based on the intensity values of the pixels (or voxels) in the second PET scan 210b depicting a matching lesion. For instance, in some cases, each matching lesion in the matched tumor mask 260 may be associated with a metabolic level corresponding to a mean, a median, a mode, a maximum, a minimum, or a range of the intensity values of the pixels (or voxels) depicting the matching lesion. The second metabolic level may, in some cases, correspond to a mean, a median, a mode, a maximum, a minimum, or a range of the metabolic levels of every matching lesion in the matched tumor mask 260. Alternatively, the second metabolic level may correspond to an average metabolic level across every matching lesion in the matched tumor mask 260 weighted by the size (e.g., volume, diameter, and/or the like) of the matching lesion.

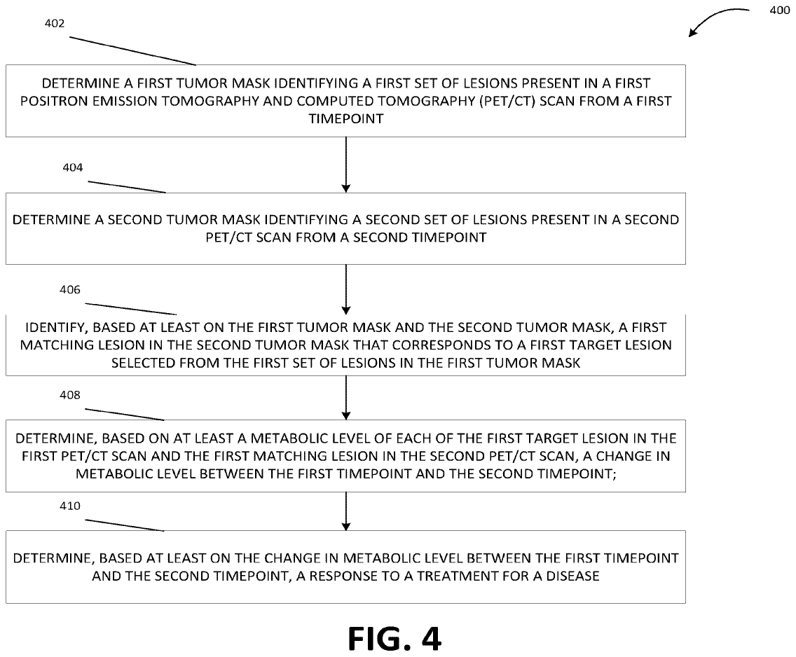

FIG. 4 depicts a flowchart illustrating an example of a process 400 for machine learning enabled determination of disease progress and treatment response based on medical imaging data, in accordance with some example embodiments. Referring to FIGS. 1-4, the process 400 may be performed by the analysis controller 110. The analysis controller 110 may perform the process 400 to determine, for example, the treatment response 180. For example, in some cases, the process 400 may be performed to determine the treatment response 180 based on the change in metabolic level exhibited at patient follow-up by lesions identified as clinically significant (e.g., due to metabolic activity, size, and/or the like) at the initial screening or rebaselining of the patient. That is, instead of tracking metabolic change indiscriminately across every lesion present at a first timepoint and every lesion present at a second timepoint, the process 400 may be performed to track the metabolic change present in certain lesions, such as those with clinical significance. For instance, in some cases, metabolic change may be tracked for lesions that are present at both the first timepoint and the second timepoint and whose metabolic level and/or size satisfy one or more thresholds. Lesions that are too small or exhibits too little metabolic activities may lack clinical significance. Moreover, lesions that are present at one but not both timepoints may be categorized as either new lesions or disappearing lesions, whose clinical significance is different than that of lesions present at both timepoints.

In doing so, the process 400 may be performed to enable a correct identification of new lesions, disappearing lesions, and existing lesions, each of which being associated with different clinical significance, such as treatment response. Moreover, the process 400 may be performed to enable a precise determination of the change in metabolic level as exhibited those existing lesions with sufficient clinical significance as indicated, for example, by metabolic level, size, and/or the like. For example, as described in more details below, an existing lesions may be a target lesion from a first timepoint with a matching lesion at a second timepoint. The target lesion may be selected (e.g., based on metabolic level, size, and/or the like) from a first set of lesions present in a first tumor mask from the first timepoint before the matching lesion is identified (e.g., based on an overlap in respective pixel coordinates) in a second tumor mask from the second timepoint as corresponding to the target lesion. Without tracking specific lesions (e.g., those exhibiting threshold metabolic level and/or size at initial screening or rebaselining) that are present between two timepoints, the determination of the treatment response 180 may be skewed by the presence of clinically insignificant lesions, which may in fact be incorrectly segmented artifacts in the underlying PET/CT scans and not actual lesions. Failure to identify new lesions and disappearing lesions may also introduce errors in the treatment response 180 as new lesions and disappearing lesions may, in some cases, be indicative of a different treatment response 180 than changes in the metabolic activities of existing lesions.

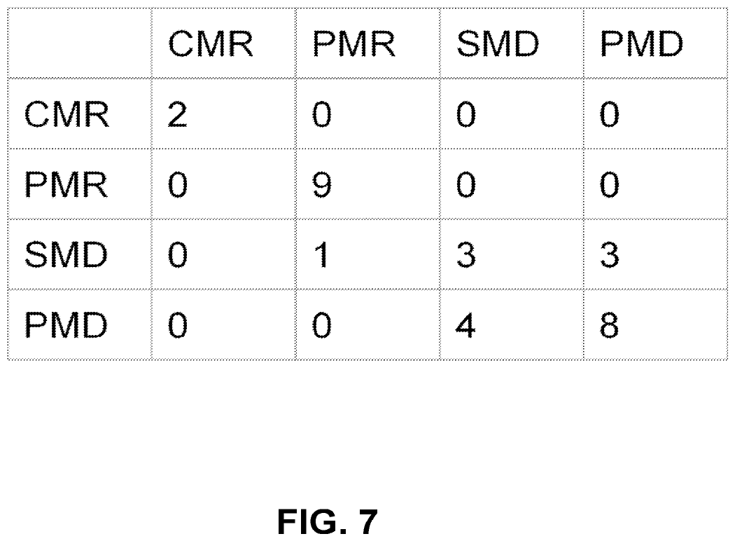

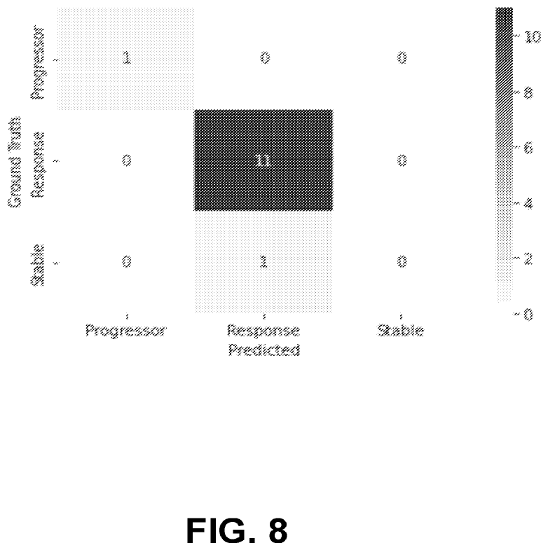

As indicated by the performance metrics shown in FIGS. 7-8, the analysis controller 110 performing the process 400 is able to achieve highly accurate results that are consistent with those determined by expert radiologists. This accuracy may be attributed, at least in part, to the exclusion of incorrectly segmented artifacts present in the underlying PET/CT scans as actual lesions when determining the treatment response 180. Given the accuracy of the treatment response 180 determined by the process 400, it should be appreciated that the treatment response 180 has a higher prognostic value than those determined by conventional techniques. Furthermore, it should be appreciated that the process 400 can be performed to achieve highly accurate results independently (e.g., without requiring expert radiologist intervention), meaning that process 400 provides a more efficient diagnostic solution that requires fewer resources than conventional techniques for analyzing positron emission tomography (PET) and computed tomography (CT) scans. The speed and efficiency of the process 400 may expedite many downstream clinical tasks including treatment decisions. In some cases, the treatment response 180 generated by the process 400 may be applied towards generating immediate treatment decisions, thereby eliminating a critical bottleneck in conventional clinical workflows.

At 402, the analysis controller 100 may determine a first tumor mask 230a identifying a first set of lesions present in a first positron emission tomography and computed tomography (PET/CT) scan from a first timepoint. In some example embodiments, the analysis controller 110 may apply a segmentation model 111 to segment the first PET/CT scan 215 and generate the first tumor mask 230a. In some cases, the first tumor mask 230a may identify one or more lesions present in the first PET/CT scan 215. For example, in some cases, each pixel in the first tumor mask 230a may be assigned a first label (e.g., having a first value such as “1”) if the pixel depicts a lesion or a second label (e.g., having a second value such as “0”) if the pixel does not depict a lesion. Moreover, in some cases, the segmentation model 111 may be coupled with (or include) a longitudinal segmentation model trained to further refine the first tumor mask 230a based on the first PET/CT scan 215 from the first timepoint, the second PET/CT scan 225 from the second timepoint, and the second tumor mask 230b identifying one or more lesions present in the second PET/CT scan 225. In some cases, the tumor selection engine 113 may select, for inclusion in the set of target lesions, one or more of the lesions present in the first tumor mask 230a. In some cases, the tumor selection engine 113 may perform its selection based on a metabolic level and/or a size (e.g., volume, diameter, and/or the like) of each lesion in the first tumor mask 230a.

At 404, the analysis controller 110 may determine a second tumor mask identifying a second set of lesions present in a second PET/CT scan from a second timepoint. In some example embodiments, the analysis controller 110 may apply the segmentation model 111 to segment the second PET/CT scan 225 and generate the second tumor mask 230b. In some cases, the second tumor mask 230b may identify one or more lesions present in the second PET/CT scan 225. For example, in some cases, each pixel in the second tumor mask 230b may be assigned a first label (e.g., having a first value such as “1”) if the pixel depicts a lesion or a second label (e.g., having a second value such as “0”) if the pixel does not depict a lesion. In some cases, the segmentation model 111 may be coupled with (or include) a longitudinal segmentation model that refines the second tumor mask 230b based on the first PET/CT scan 215, the first tumor mask 230a, and the second PET/CT scan 225.

At 406, the analysis controller 110 may identify, based at least on the first tumor mask and the second tumor mask, a matching lesion in the second tumor mask that corresponds to a target lesion selected from the first set of lesions in the first tumor mask. In some example embodiments, the tumor matching engine 117 may identify, based at least on an overlap between a first plurality of pixel coordinates of each target lesion in the first tumor mask 230a (e.g., the selected tumor mask and a second plurality of aligned pixel coordinates of each lesion in the second tumor mask 230b aligned with the first tumor mask 230a (e.g., the aligned tumor mask), one or more matching lesions in the second tumor mask 230b that corresponds to the target lesions in the first tumor mask 230a. In some cases, prior to tumor matching, the alignment engine 115 may align the first tumor mask 230a and the second tumor mask 230b such that a first fiducial marker (e.g., a first anatomical landmark) in the first tumor mask 230a is aligned with a second fiducial marker (e.g., a second anatomical landmark) in the second tumor mask 230b. The alignment of the first tumor mask 230a and the second tumor mask 230b may include applying, to the unaligned pixel coordinates in the second tumor mask 230b, one or more transformations (e.g., affine transformations and/or the like) that align the second fiducial marker (e.g., the second anatomical landmark) in the second tumor mask 230b with the first fiducial marker (e.g., the first anatomical landmark) in the first tumor mask 230a. Doing so may generate the aligned tumor mask 250, which is used by the tumor matching engine 117 to perform tumor matching and identify, within the aligned tumor mask 250, one or more matching lesions that correspond to the target lesions in the first tumor mask 230a.

In some example embodiments, the tumor matching engine 117 may perform tumor matching by at least determining the overlap between a first plurality of pixel coordinates of a target lesion in the first tumor mask 230a and a second plurality of aligned pixel coordinates of a lesion in the aligned tumor mask 250. In some cases, this overlap may correspond to an intersection-over-union (IOU) between the first plurality of pixel coordinates and the second plurality of aligned pixel coordinates. Moreover, in some cases, the tumor matching engine 117 may identify the lesion in the aligned tumor mask 250 as a matching lesion corresponding to the target lesion in the first tumor mask 230a if the intersection-over-union (IOU) between the corresponding pixel coordinates satisfies one or more thresholds. In some cases, the tumor matching engine 117 may generate the matched tumor mask 260 to identify one or more matching lesions identified within the aligned tumor mask 250. For example, the matched tumor mask 260 may include pixels (or voxels) depicting a matching lesion being assigned a first label (e.g., having a first value such as “1”) and pixels (or voxels) not depicting a matching lesion being assigned a second label (e.g., having a second value such as “0”). It should be appreciated that the tumor matching process may be iterative in that the tumor matching engine 117 may iterate through every target lesion selected from the first tumor mask 230a and determine, for each target lesion, an overlap with each lesion in the aligned tumor mask 250 until either (i) a matching lesion having a threshold overlap is identified or (ii) the tumor matching engine 117 has examined every lesion in the second tumor mask 230b.

At 408, the analysis controller 110 may determine, based on at least a metabolic level of each of the target lesion in the first PET/CT scan and the matching lesion in the second PET/CT scan, a change in metabolic level between the first timepoint and the second timepoint. In some example embodiments, the assessment engine 119 may determine the difference between a first metabolic level at the first timepoint and a second metabolic level at the second timepoint. For example, in some cases, the first metabolic level at the first timepoint may be determined based on the metabolic level of each target lesion selected from the first tumor mask 230a (e.g., the selected tumor mask 240) while the second metabolic level at the second timepoint may be determined based on the metabolic level of each matching lesion in the matched tumor mask 260. As noted, the metabolic level of an individual lesion may correspond to a mean, a median, a mode, a maximum, a minimum, or a range of the intensity values of the pixels (or voxels) depicting the lesion in a PET scan. Meanwhile, the metabolic level at a single timepoint may correspond a mean, a median, a mode, a maximum, a minimum, or a range of the metabolic levels of the lesions present at the timepoint. In some cases, the metabolic level at a single timepoint may correspond to an average metabolic level of the lesions present at that timepoint weighted by the size (e.g., volume, diameter, and/or the like) of each lesion. Where multiple lesions of varying sizes (e.g., volume, diameter, and/or the like) may be present at any one timepoint, determining a metabolic level in the foregoing manner may generate a single metric that captures the overall metabolic level across different lesions.

At 410, the analysis controller may determine, based at least on the change in metabolic level between the first timepoint and the second timepoint, a response to a treatment for a disease. As will be described in more detail below, the assessment engine 119 may determine the treatment response 180 to be one of progressive metabolic disease (PMD), stable metabolic disease (SMD), and partial metabolic response (SMR) based at least on whether the change in metabolic level between the first timepoint and the second timepoint satisfies the one or more thresholds. For instance, the assessment engine 119 may determine that the change in metabolic level is increasing if the change in metabolic level satisfies a first threshold and decreasing if the change in metabolic level fails to satisfy the first threshold. The assessment engine 119 may determine whether the change in metabolic level exhibits a threshold increase (or threshold decrease) depending on whether the change in metabolic level further satisfies a second threshold.

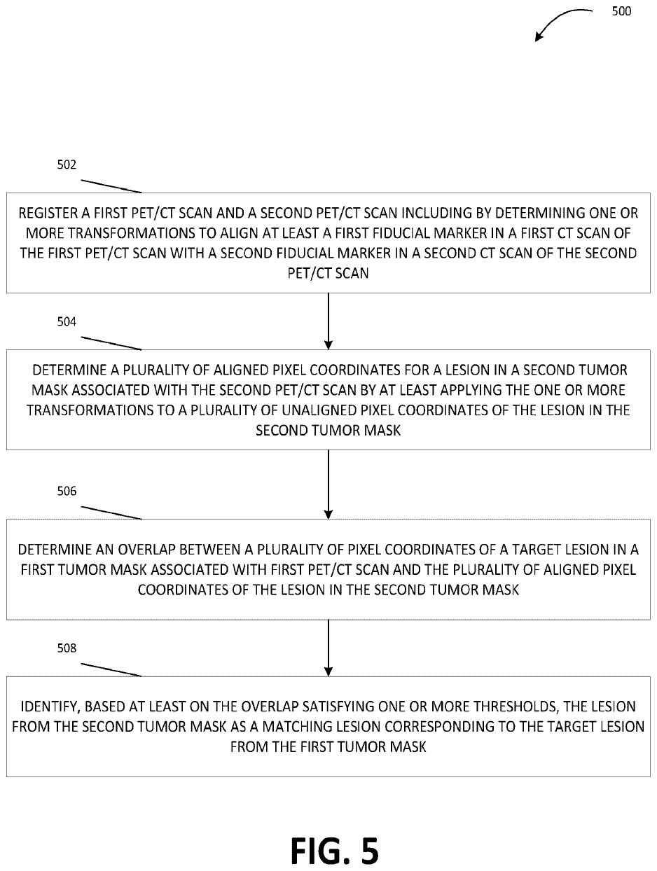

FIG. 5 depicts a flowchart illustrating another example of a process 500 for tumor matching, in accordance with some example embodiments. Referring to FIGS. 1-5, the process 500 may be performed by the analysis controller 110 and may implement, for example, operation 406 of the process 400. In some cases, the process 500 for tumor matching may be iterative, meaning that the analysis controller 500 may repeat at least a portion of the process 500 for each pair of lesions in the first tumor mask 230a and the second tumor mask 230b. For example, in some cases, at least a portion of the process 500 may be performed iteratively, for each target lesion selected from the first tumor mask 230a, until either (i) a matching lesion having a threshold overlap is identified in the second tumor mask 230b or (ii) the tumor matching engine 117 has examined every lesion in the second tumor mask 230b.

In some cases, the process 500 may be performed to identify lesions that are present at both the first timepoint of the first tumor mask 230a and the second timepoint of the second tumor mask 230b. Clinical assessments, such as the determination of the treatment response 180, may be made based on changes in the metabolic activity of lesions between timepoints but the accuracy and precision of these clinical assessments may depend on correctly identifying the same lesions across the first tumor mask 230a and the second tumor mask 230b. This is a nontrivial task at least because a lesion may undergo significant morphological changes between the first timepoint of the first tumor mask 230a and the second timepoint of the second tumor mask 230 such that the lesion in the first tumor mask 230a appears dissimilar to the same lesion in the second tumor mask 230. Furthermore, the first tumor mask 230a and the second tumor mask 230b may not be sufficiently aligned due to misalignment between the underlying first PET/CT scan 215 and second PET/CT scan 225. Accordingly, as described in more details below, in addition to aligning the first tumor mask 230a and the second tumor mask 230b, the process 500 may include each of the lesions present in the first tumor mask 230a being matched against the lesions in the second tumor mask 230b. The process 500 may be advantageous at least because the process 500 may be performed to enable an accurate and precise identification of the same lesions that appear in both the first tumor mask 230a and the second tumor mask 230b. In some cases, the process 500 may also be performed to detect disappearing lesions, which are those lesions in the first tumor mask 230a that are no longer present in the second tumor mask 230b.

At 502, the analysis controller 110 may register a first PET/CT scan and a second PET/CT scan including by determining one or more transformations to align at least a first fiducial marker in a first CT scan of the first PET/CT scan with a second fiducial marker in a second CT scan of the second PET/CT scan. In some example embodiments, the alignment engine 115 may register the first PET/CT scan 215 from the first timepoint and the second PET/CT scan 225 from the second timepoint based at least on the constituent first CT scan 220a and second CT scan 220b. For example, in some cases, the alignment engine 115 may perform the registration by at least determining one or more transformations (e.g., affine transformations) to align a first fiducial marker (e.g., a first anatomical landmark) in the first CT scan 220a with a second fiducial marker (e.g., a second anatomical landmark) in the second CT scan 220b.

At 504, the analysis controller 110 may determine a plurality of aligned pixel coordinates for a lesion in a second tumor mask associated with the second PET/CT scan by at least applying the one or more transformations to a plurality of unaligned pixel coordinates of the lesion in the second tumor mask. In some example embodiments, the alignment engine 115 may apply, to the unaligned pixel coordinates in the second tumor mask 230b identifying one or more lesions present in the second PET/CT scan 225, the one or more transformations (e.g., affine transformations and/or the like) determined, for example, at operation 502, to align the first fiducial marker (e.g., the first anatomical landmark) in the first CT scan 220a with the second fiducial marker (e.g., the second anatomical landmark) in the second CT scan 220b. Doing so may generate an aligned tumor mask 250 whose pixel coordinates are aligned with the pixel coordinates in the first tumor mask 230a. Moreover, in some cases, applying the one or more transformations may transform, into aligned pixel coordinates for subsequent tumor matching between the first tumor mask 230a and the second tumor mask 230b, the unaligned pixel coordinates of the individual lesions present in the second tumor mask 230b.

At 506, the analysis controller 110 may determine an overlap between a plurality of pixel coordinates of a target lesion from a first tumor mask associated with the first PET/CT scan and the plurality of aligned pixels coordinates of the lesion in the second tumor mask. In some example embodiments, the tumor matching engine 117 may determine the overlap between a target lesion in the first tumor mask 230a and the lesion in the second tumor mask 230b by at least determining an intersection-over-union (IOU) between the pixel coordinates of the target lesion in the first tumor mask 230a (e.g., the selected tumor mask 240) and the aligned pixel coordinates of the lesion from the second tumor mask 230b (e.g., the aligned tumor mask 250).

At 508, the analysis controller 117 may identify, based at least on the overlap satisfying one or more thresholds, the lesion in the second tumor mask as a matching lesion corresponding to the target lesion from the first tumor mask. In some example embodiments, the tumor matching engine 117 may determine that the lesion from the second tumor mask 230b is a matching lesion that corresponds to the target lesion selected from the first tumor mask 230a if the intersection-over-union (IOU) between the pixel coordinates of the target lesion in the first tumor mask 230a (e.g., the selected tumor mask 240) and the aligned pixel coordinates of the lesion from the second tumor mask 230b (e.g., the aligned tumor mask 250) satisfies one or more threshold. Alternatively, where the intersection-over-union (IOU) between the pixel coordinates fails the one or more thresholds, the tumor matching engine 117 may that the lesion from the second tumor mask 230b is not a matching lesion. As noted, since tumor matching may be an iterative process, in cases where the lesion in the second tumor mask is identified as a matching lesion, the analysis controller 117 may repeat at least a portion of the process 500 for another target lesion selected from the first tumor mask 230a (e.g., the selected tumor mask 240) until every target lesion from the first tumor mask 230a (e.g., the selected tumor mask 240) has been examined. In cases where the lesion from the second tumor mask 230 is not a matching lesion, the analysis controller 117 may repeat at least a portion of the process 500 for another lesion in the second tumor mask 230b or, upon having examined every lesion in the second tumor mask 230b, continue to repeat at least a portion of the process 500 for another target lesion in the first tumor mask 230a.

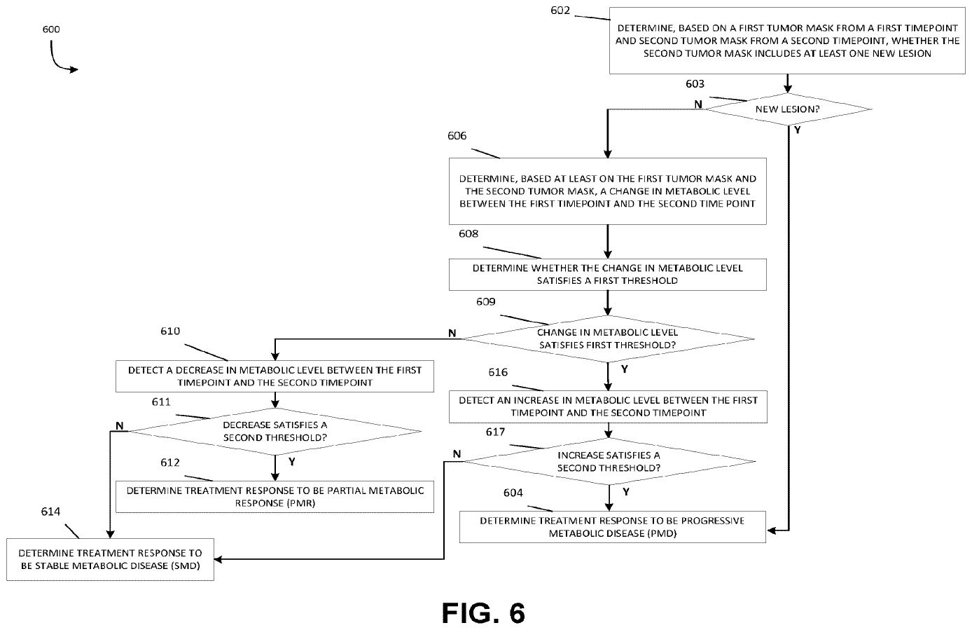

FIG. 6 depicts a flowchart illustrating an example of a process 600 for determination of treatment response, in accordance with some example embodiments. Referring to FIGS. 1-6, the process 600 may be performed by the analysis controller 110 and may implement, for example, operations 408 and 410 of the process 400. In some cases, the process 600 may implement an European Organization for Research and Treatment of Cancer (EORTC) classification process. Accordingly, in some cases, the process 600 may derive its prognostic value from monitoring the change in metabolic level across two (or more) different timepoints of certain target lesions that satisfy certain criteria with respect to metabolic level, size (e.g., volume, diameter), and/or the like. For example, the treatment response 180 determined by the performance of the process 600 may correspond to the change in metabolic level exhibited at patient follow-up by those lesions identified as clinically significant at the initial screening or rebaselining of the patient. In some cases, a lesion may be identified as clinically significant based on criteria such as its level of metabolic activity, size, and/or the like. Moreover, in some cases, the development of a new lesion at a subsequent timepoint may have a different clinical significance than changes in the metabolic activity of one or more existing lesions. The process 600 is advantageous at least because the process 600 may be performed to differentiate between the clinical significance that is associated with new lesions, an increase in the metabolic activity of existing lesions, and a decrease in the metabolic activity of existing lesions.

As described in more details below, in some cases, the treatment response 180 may be determined based on a change in metabolic level between a target lesion selected from a first set of lesions present in the first tumor mask 230a from a first timepoint and a matching lesion identified (e.g., based on an overlap in respective pixel coordinates) in the second tumor mask 230b from a second timepoint as corresponding to the target lesion. Moreover, the treatment response 180 that is generated by the analysis controller 110 may conform to the EORTC criteria for treatment response assessment, which includes determinations of complete metabolic response (CMR), progressive metabolic disease (PMD), stable metabolic disease (SMD), or partial metabolic response (PMR) made based on a patient's PET/CT scans from two (or more) different timepoints, such as initial patient screening or rebaselining and follow-up. The treatment response 180 generated by the analysis controller 110 may exhibit the granularity required to conform to the EORTC criteria at least because the process 600 may be performed to differentiate between the development of new lesions, increases in the metabolic activity of existing lesions, and decreases in the metabolic activity of existing lesions.

The performance metrics shown in FIGS. 7-8 indicate that the analysis controller 110 performing the process 600 is able to achieve highly accurate results. That is, the EORTC classification resulting from the analysis controller 110 performing the process 600 are consistent with the determinations made by expert radiologists. Furthermore, the process 600 can be performed to achieve highly accurate results independently (e.g., without requiring expert radiologist intervention) and is therefore more expedient, efficient, and requires fewer resources than conventional techniques for analyzing PET/CT scans. The speed and efficiency of the process 600 may expedite many downstream clinical tasks including treatment decisions. In some cases, the treatment response 180 generated by the process 600 may be applied towards generating immediate treatment decisions, thereby eliminating a critical bottleneck in conventional clinical workflows.

As described in more details below, the process 600, which leverages insights derived from machine learning enabled analysis of medical imaging data such as PET/CT scans, can be performed to generate the more granular EORTC classifications than conventional techniques. For example, the process 600 may be performed to provide an accurate and precise differentiation between patients exhibiting various treatment responses, such as progressive metabolic disease (PMD), stable metabolic disease (SMD), complete metabolic response (CMR), and partial metabolic response (PMR), which may be more insightful than a binary classification (e.g., complete metabolic response (CMR) vs. non-complete metabolic response (Non-CMR)). The accuracy and precision of the treatment response 180 generated by the process 600 means that the process 600 also improves the accuracy and precision of downstream clinical tasks that rely on the outputs of the process 600 including, for example, the identification of relapse and refractory patients, treatment planning, and/or the like.

At 602, the analysis controller 110 may determine, based on a first tumor mask from a first timepoint and a second tumor mask from a second timepoint, whether the second tumor mask includes at least one new lesion. For example, in some cases, the tumor matching engine 117 may perform tumor matching based on the selected tumor mask 240 identifying a set of target lesions selected from the first tumor mask 230a (e.g., based on metabolic level and/or size) and the aligned tumor mask 250 identifying one or more lesions present in the second tumor mask 230b aligned with the first tumor mask 230a. In instances where the second tumor mask 230b is determined to include one or more lesions that fail to correspond to any of the target lesions selected from the first tumor mask 230a, the assessment engine 119 may determine that the second tumor mask 230b includes one or more new lesions. Contrastingly, the second tumor mask 230b may not include any new lesions if every lesion present in the second tumor mask 230b is determined to correspond to a target lesion selected from the first tumor mask 230a.

At 603-Y, the analysis controller 110 may determine that the second tumor mask includes at least one new lesion. Accordingly, at 604, the analysis controller 110 may determine that the treatment response to be progressive metabolic disease (PMD). For example, in instances where the second tumor mask 230b is determined to include one or more lesions that fail to correspond to any of the target lesions selected from the first tumor mask 230a, the assessment engine 119 may determine that the treatment response 180 is progressive metabolic disease (PMD).

Alternatively, at 603-N, the analysis controller 110 may determine that the second tumor mask does not include at least one new lesion. As such, the process 600 may resume with the analysis controller 110 may determining a change in metabolic level between lesions that are present at the first timepoint as well as the second timepoint. For example, at 606, the analysis controller 110 may determine, based at least on the first tumor mask and the second tumor mask, a change in metabolic level between the first timepoint and the second timepoint. In some cases, the analysis controller 110 may determine the change in metabolic level between the first timepoint and the second timepoint by at least determining a difference between a first metabolic level exhibited by the target lesions selected from the first tumor mask 230a and a second metabolic level exhibited by the matching lesions identified within the second tumor mask 230b. In some cases, the first metabolic level at the first timepoint may correspond to a mean, a mode, a median, a maximum, a minimum, or a range of the metabolic level of each target lesion selected from the first tumor mask 230a while the second metabolic level at the second timepoint may correspond to a mean, a mode, a median, a maximum, a minimum, or a range of the metabolic level of each matching lesion present in the second tumor mask 230b. Alternatively, in some cases, the first metabolic level may correspond to an average metabolic level of the target lesions selected from the first tumor mask 230a weighted by size (e.g., volume, diameter, and/or the like) while the second metabolic level may corresponding to an average metabolic level of the matching lesions in the second tumor mask 230b weighted by size (e.g., volume, diameter, and/or the like). To further illustrate, the change in metabolic level ΔP between the first timepoint t₁ and the second timepoint t₂ may be determined based on Equations (1) and (2) below.

$$Δ P = \frac{{p}_{t 2} - {p}_{t 1}}{{p}_{t 1}} \qquad ( 1 )$$

$${P}_{tx} = \frac{{\sum }_{i = 1}^{n} {w}_{i} {SUV}_{i * t x}}{{\sum }_{t = 1}^{n} {w}_{i}} \qquad ( 2 )$$

wherein pₜ₁ denotes the first metabolic level at the first timepoint t₁ (e.g., screening, rebaselining, prior to treatment, and/or the like) and pₜ₂ denotes the second metabolic level at the second timepoint t₂ (e.g., follow-up, subsequent to treatment, and/or the like). According to Equation (2), the metabolic level pₜ_{ₓ} at any timepoint tₓ may correspond to an average of the metabolic level SUVᵢ₊ₜₓ of each lesion i present at the timepoint tₓ weighted by the corresponding size wᵢ (e.g., volume, diameter, and/or the like).

At 608, the analysis controller 110 may determine whether the change in metabolic level between the first timepoint and the second timepoint satisfies a first threshold. For example, in some cases, the assessment engine 119 may determine, based at least on whether the change in metabolic level between the first timepoint and the second timepoint satisfies a first threshold, if the metabolic level is increasing or decreasing between the first timepoint and the second timepoint. Accordingly, at 609-N, the analysis controller 110 may determine that the change in metabolic level fails to satisfy the first threshold. At 610, upon determining that the change in metabolic level between the first timepoint and the second timepoint fails to satisfy the first threshold, the analysis controller 110 may detect a decrease in metabolic level between the first timepoint and the second timepoint. Alternatively, at 609-Y, the analysis controller 110 may determine that the change in metabolic level satisfies the first threshold. When that is the case, at 616, the analysis controller 110 may detect an increase in metabolic level between the first timepoint and the second timepoint.

Returning to operation 610, upon detecting a decrease in metabolic level between the first timepoint and the second timepoint, the analysis controller 110, for example, the assessment engine 119, may determine the treatment response 180 based on whether that decrease in metabolic level satisfies a second threshold. Accordingly, at 611-Y, the analysis controller 110 may determine that the decrease in metabolic level between the first timepoint and the second timepoint satisfies a second threshold. In response to the decrease in metabolic level satisfying the second threshold, at 612, the analysis controller 110 may determine that the treatment response to be partial metabolic response (PMR). Alternatively, at 611-N, the analysis controller 110 may determine that the decrease in metabolic level between the first timepoint and the second timepoint fails to satisfy the second threshold. When that is the case, at 614, the analysis controller 110 may determine the treatment response to be stable metabolic disease (SMD).

Returning to operation 616, upon detecting an increase in metabolic level between the first timepoint and the second timepoint, the analysis controller 110, for example, the assessment engine 119, may determine the treatment response 180 based on whether the increase in metabolic level satisfies a second threshold. As such, at 617-Y, the analysis controller 110 may determine that the increase in the metabolic activity between the first timepoint and the second timepoint satisfies the second threshold. At 604, in response to the increase in metabolic activity satisfying the second threshold, the analysis controller 110 may determine the treatment response to be progressive metabolic disease (PMD). Alternatively, at 617-N, the analysis controller 110 may determine that the increase in metabolic activity between the first timepoint and the second timepoint fails to satisfy the second threshold. In response to the increase in metabolic activity failing to satisfy the second threshold, at 614, the analysis controller 110 may determine the treatment response to be stable metabolic disease (SMD).

As noted, the analysis controller 110 may determine the treatment response 180 more efficiently and with a high level of accuracy. The performance of the analysis controller 110 may be evaluated based on datasets from different populations and treatment protocols. FIG. 7 shows the accuracy and validation of the analysis controller 110 on an example experiment involving 30 baseline-follow scans in a breast cancer trial evaluating using the EORTC criteria. The columns in the table include the treatment responses determined by the analysis controller 110 while the rows in the table include the ground truth treatment responses determined by expert radiologists. For example, in the first row corresponding to the two PET/CT scans with the ground-truth treatment response complete metabolic response (CMR), the analysis controller 110 correctly predicted the treatment response complete metabolic response (CMR) for both PET/CT scans. In the second row corresponding the nine PET/CT scans having the ground-truth treatment response partial metabolic response (PMR), the analysis controller 110 was able to correctly predict the correct treatment response for all nine PET/CT scans. In row three, which corresponds to PET/CT scans with the ground-truth treatment response of stable metabolic disease, the analysis controller 110 was able to determine the correct treatment response for three out of the seven PET/CT scans in that category. In the fourth row, which corresponds to PET/CT scans having the ground-truth treatment response of progressive metabolic disease (PMD), the analysis controller 110 was able to determine the correct treatment response for eight out of the twelve PET/CT scans in that category. In this example experiment, the analysis controller 110 is able to achieve an overall accuracy of 73.3%, meaning that 73.3% of the treatment responses predicted by the analysis controller 110 match that determined by an expert radiologist. In particular, the analysis controller 110 was able to determine the correct treatment response for every PET/CT scan exhibiting either complete metabolic response (CMR) or partial metabolic response (PMR).

FIG. 8 depicts another example experiment involving 12 patients in a melanoma study. In the table shown in FIG. 8, the category “stable” corresponds to stable metabolic response (SMR), the category “progressor” corresponds to progressive metabolic disease (SMD), and the category “response” covers partial metabolic response (PMR) and complete metabolic response (CMR). The analysis controller 110 performed with very high accuracy in this experiment, with 92.3% (or eleven out of twelve) of the treatment responses determined by the analysis controller 110 matching that determined by expert radiologists. For example, as shown in FIG. 8, the analysis controller 110 determined the correct treatment response for the one PET/CT scan in the “progressor” category and all eleven PET/CT scans in the “response” category.

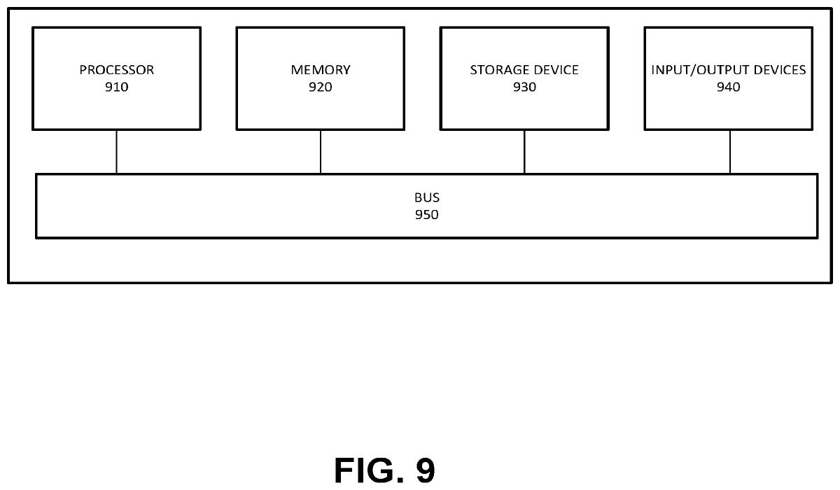

FIG. 9 depicts a block diagram illustrating an example of a computing system 900 consistent with implementations of the current subject matter. Referring to FIGS. 1-9, the computing system 900 can be used to implement the analysis controller 110, the one or more imaging device 120, the client device 130, and/or any components therein.

As shown in FIG. 9, the computing system 900 can include a processor 910, a memory 920, a storage device 930, and an input/output device 940. The processor 910, the memory 920, the storage device 930, and the input/output device 940 can be interconnected via a system bus 950. The processor 910 is capable of processing instructions for execution within the computing system 900. Such executed instructions can implement one or more components of, for example, the analysis controller 110, the one or more imaging devices 120, and the client device 130. In some example embodiments, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a solid state drive, a floppy disk device, a hard disk device, an optical disk device, or a tape device, or other suitable persistent storage means. The input/output device 940 provides input/output operations for the computing system 900. In some example embodiments, the input/output device 940 includes a keyboard and/or pointing device. In various implementations, the input/output device 940 includes a display unit for displaying graphical user interfaces.

According to some example embodiments, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the Internet).

In some example embodiments, the computing system 900 can be used to execute various interactive computer software applications that can be used for organization, analysis and/or storage of data in various formats. Alternatively, the computing system 900 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT), a liquid crystal display (LCD), a light emitting diode (LED) monitor, or an organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
determining a baseline tumor mask identifying a baseline lesion set present in a baseline positron emission tomography and computed tomography (PET/CT) scan from a first timepoint;
determining a followup tumor mask identifying a followup lesion set present in a followup PET/CT scan from a second timepoint;
identifying, based at least on the baseline tumor mask and followup tumor mask, a matching lesion in the followup tumor mask that corresponds to a target lesion selected from the baseline lesion set in the baseline tumor mask, the matching lesion being identified based at least on an overlap between a plurality of pixel coordinates of the target lesion in the baseline tumor mask and a plurality of aligned pixel coordinates of the matching lesion in the followup tumor mask aligned with the baseline tumor mask;
determining, based on at least a metabolic level of each of the target lesion in the baseline PET/CT scan and the matching lesion in the followup PET/CT scan, a change in metabolic level between the first timepoint and the second timepoint; and
determining, based at least on the change in metabolic level between the first timepoint and the second timepoint, a response to a treatment for a disease.

2. (canceled)

3. (canceled)

4. The system of claim 1, further comprising:
registering a baseline CT scan in the baseline PET/CT scan and a followup CT scan in the followup PET/CT scan by at least
determining one or more transformations to align at least a first fiducial marker in the baseline PET/CT scan with a second fiducial marker in the followup PET/CT scan; and
in response to determining that the baseline tumor mask includes at least the target lesion and the followup tumor mask includes at least the matching lesion, applying the one or more transformations to a plurality of unaligned pixel coordinates of the matching lesion in the followup tumor mask in order to generate the plurality of aligned pixels coordinates of the matching lesion in the followup tumor mask aligned with the baseline tumor mask.

5. The system of claim 4, further comprising:
determining the overlap between the first plurality of pixel coordinates of the target lesion and the plurality of aligned pixel coordinates of the matching lesion by at least determining an intersection-over-union (IOU) between the plurality of pixel coordinates and the plurality of aligned pixel coordinates; and
identifying, based at least on the intersection-over-union (IOU) satisfying one or more thresholds, the matching lesion from the second timepoint as corresponding to the target lesion from the first timepoint.

6. (canceled)

7. The system of claim 4, further comprising:
in response to determining that the baseline tumor mask includes at least the target lesion but the followup tumor mask fails to include any lesions, determining that the response to the treatment for the disease is complete metabolic response (CMR); and
in response to determining that the baseline tumor mask fails to include any lesions and the followup tumor mask includes at least the matching lesion, determining that the response to the treatment for the disease is progressive metabolic disease (PMD).

8. (canceled)

9. The system of claim 1, further comprising:
identifying, based at least on the baseline tumor mask and the followup tumor mask, a matching lesion for every target lesion selected from the baseline lesion set, wherein the identifying includes determining, for each target lesion selected from the baseline lesion set, an overlap with each lesion in the followup lesion set.

10. The system of claim 1, further comprising:
identifying, based at least on the baseline tumor mask and the followup tumor mask, at least one new lesion in the followup lesion set that fails to match any target selections selected from the baseline lesion set; and
in response to identifying the at least one new lesions in the followup lesion set, determining the response to the treatment as progressive metabolic disease (PMD).

11. The system of claim 1, further comprising:
determining, based at least on a metabolic level of the target lesion, an overall metabolic level of the baseline lesion set at the first timepoint;
determining, based at least on a metabolic level of the matching lesion, an overall metabolic level of the followup lesion set at the second timepoint; and
determining, based at least on a difference between the overall metabolic level at the first timepoint and the overall metabolic level at the second timepoint, the change in metabolic level between the first timepoint and the second timepoint.

12. (canceled)

13. (canceled)

14. The system of claim 11, further comprising:
determining, based at least on a metabolic level of each constituent pixel in a corresponding PET scan, the metabolic level of each of the target lesion and the matching lesion.

15. (canceled)

16. (canceled)

17. The system of claim 1, wherein the response to the treatment is determined as progressive metabolic disease (PMD) based at least on the change in metabolic level satisfying a first threshold.

18. The system of claim 17, wherein the response to the treatment is determined as stable metabolic disease (SMD) based at least on the change in metabolic level satisfying a second threshold but failing to satisfy the first threshold.

19. The system of claim 18, wherein the response to the treatment is determined as partial metabolic response (PMR) based at least on the change in metabolic level failing to satisfy the first threshold and the second threshold.

20. The system of claim 1, wherein the response to the treatment for the disease is determined by at least
determining, based at least on the change in metabolic level between the first timepoint and the second timepoint satisfying a first threshold, that a metabolic level exhibits a threshold increase between the first timepoint and the second timepoint; and
in response to determining that the metabolic level exhibits the threshold increase, determining that the response to the treatment for the disease is progressive metabolic disease (PMD).

21. The system of claim 20, wherein the response to the treatment for the disease is further determined by at least
determining, based at least on the change in metabolic level between the first timepoint and the second timepoint satisfying a second threshold, that the metabolic level exhibits a threshold decrease between the first timepoint and the second timepoint; and
in response to determining that the metabolic level exhibits the threshold decrease, determining that the response to the treatment for the disease is partial metabolic response (PMR).

22. The system of claim 21, wherein the response to the treatment for the disease is further determined by at least
determining, based at least on the change in metabolic level between the first timepoint and the second timepoint failing to satisfy the first threshold and the second threshold, that the metabolic level fails to exhibit the threshold increase and the threshold decrease between the first timepoint; and
in response to determining that the metabolic level fails to exhibit the threshold increase and the threshold decrease, determining that the response to the treatment for the disease is stable metabolic disease (SMD).

23. The system of claim 1, further comprising:
determining, based at least on the baseline tumor mask, at least one of a metabolic level and a size of each lesion in the baseline lesion set depicted in the baseline PET/CT scan; and
selecting, based on the at least one of the metabolic level and the size of each lesion in the baseline lesion set, the target lesion for inclusion in a set of target lesions.

24. The system of claim 23, wherein the target lesion is selected for inclusion in the set of target lesions based at least on the metabolic level and/or the size of the target lesion satisfying one or more thresholds.

25. The system of claim 23, wherein the target lesion is selected for inclusion in the set of target lesions based at least on the target lesion being one of a threshold quantity of lesions having a highest metabolic level and/or a largest size within the baseline lesion set.

26. The system of claim 1, wherein the baseline PET/CT scan is performed prior to the treatment of the disease, and wherein the followup PET/CT scan is performed subsequent to the treatment for the disease.

27. (canceled)

28. A computer-implemented method, comprising:
determining a baseline tumor mask identifying a baseline lesion set-present in a baseline positron emission tomography and computed tomography (PET/CT) scan from a first timepoint;
determining a followup tumor mask identifying a followup lesion set present in a followup PET/CT scan from a second timepoint;
identifying, based at least on the baseline tumor mask and followup tumor mask, a matching lesion in the followup tumor mask that corresponds to a target lesion selected from the baseline lesion set in the baseline tumor mask, the matching lesion being identified based at least on an overlap between a plurality of pixel coordinates of the target lesion in the baseline tumor mask and a plurality of aligned pixel coordinates of the matching lesion in the followup tumor mask aligned with the baseline tumor mask;
determining, based on at least a metabolic level of each of the target lesion in the baseline PET/CT scan and the matching lesion in the followup PET/CT scan, a change in metabolic level between the first timepoint and the second timepoint; and
determining, based at least on the change in metabolic level between the first timepoint and the second timepoint, a response to a treatment for a disease.

29. A non-transitory computer readable medium storing instructions, which when executed by the at least one data processor, result in operations comprising:
determining a baseline tumor mask identifying a baseline lesion set-present in a baseline positron emission tomography and computed tomography (PET/CT) scan from a first timepoint;
determining a followup tumor mask identifying a followup lesion set present in a followup PET/CT scan from a second timepoint;
identifying, based at least on the baseline tumor mask and followup tumor mask, a matching lesion in the followup tumor mask that corresponds to a target lesion selected from the baseline lesion set in the baseline tumor mask, the matching lesion being identified based at least on an overlap between a plurality of pixel coordinates of the target lesion in the baseline tumor mask and a plurality of aligned pixel coordinates of the first matching lesion in the followup tumor mask aligned with the baseline tumor mask;
determining, based on at least a metabolic level of each of the target lesion in the baseline PET/CT scan and the matching lesion in the followup PET/CT scan, a change in metabolic level between the first timepoint and the second timepoint; and
determining, based at least on the change in metabolic level between the first timepoint and the second timepoint, a response to a treatment for a disease.

30. (canceled)

31. (canceled)

32. (canceled)
